(19) United States
(12) Patent Application Publication
(10) Pub. No.: US 2026/0159813 A1
(43) Pub. Date: Jun. 11, 2026

(54) CULTURE MEDIA AND CONDITIONS FOR IN VITRO EXPANSION AND/OR MATURATION OF HEPATOCYTES

(71) Applicant: Satellite Biosciences, Inc., Cambridge, MA (US)

(72) Inventors: Sunil Kumar MALLANNA, Natick, MA (US); Maxwell P. VAN BUSKIRK, Somerville, MA (US); Soujanya S. KARANTH, Natick, MA (US)

(21) Appl. No.: 18/704,122

(22) PCT Filed: Oct. 25, 2022
(86) PCT No.: PCT/US2022/047753

§ 371 Date: Apr. 24, 2024

Related U.S. Application Data
(60) Provisional application No. 63/271,441, filed on Oct. 25, 2021.

(51) Int. Cl. C12N 5/071

(52) U.S. Cl. CPC C12N 5/067; C12N 2500/02; C12N 2500/32; C12N 2501/01; C12N 2501/11; C12N 2501/117; C12N 2501/119; C12N 2501/12; C12N 2501/148; C12N 2501/39; C12N 2501/395; C12N 2501/40; C12N 2501/415; C12N 2533/52; C12N 2533/54

(57) ABSTRACT

Provided herein are completely defined two-dimensional culture conditions, including culture supplements and requirements for an extracellular matrix, facilitating the expansion of and long-term maintenance of primary human hepatocytes, as well as culture conditions and media for maturation of hepatocytes.

### FIELD OF THE INVENTION

The present disclosure relates generally to methods of culturing, expanding, and maturing hepatocytes.

### BACKGROUND OF THE INVENTION

Liver disease (e.g., hepatic disease) is any disease that negatively affects the normal, healthy performance of the liver. The resulting disturbance of liver function causes illness. For example, impaired liver function can result in an accumulation of toxins (e.g., nitrogenous waste compounds) in the blood. These toxins may travel to the brain and affect the nervous system. The CDC reports that 4.5 million Americans have been diagnosed with liver disease. While organ replacement therapy can rescue impaired native liver function, the demand far exceeds availability. A feasible alternative is to implant a population of primary human hepatocytes (PHH), which are in short supply.

Therefore, improved methods for culturing and expanding PHHs are needed.

### SUMMARY OF THE INVENTION

The invention provides methods for long-term maintenance, expansion, and maturation of primary human hepatocytes (PHH), which may be useful for generating grafts for implantation in human recipients to supplement or rescue native liver function.

In one aspect, the disclosure provides a method for culturing PHH, the method including the step of culturing one or more hepatocyte in contact with an extracellular matrix (ECM) in the presence of an expansion medium that includes a basal medium for human cells to which is added one or more (e.g., two, three, four, or five) Wnt signaling activators, one or more (e.g., two, three, four, or five) receptor tyrosine kinase ligands, and one or more (e.g., two, three, four, or five) epithelial phenotype stabilizing agents.

In some embodiments of the foregoing aspect, one or more (e.g., two, three, four, or five) of the Wnt signaling activators is R-spondin 1, R-spondin 2, R-spondin 3, R-spondin 4, Wnt3a, or a combination of any of the foregoing. In some embodiments, the one or more Wnt signaling activator includes R-spondin 1 and Wnt3a.

In some embodiments of the foregoing aspect, one or more (e.g., two, three, four, or five) of the receptor tyrosine kinase ligands is an epidermal growth factor (EGF), a fibroblast growth factor (FGF), a hepatocyte growth factor (HGF), a transforming growth factor (TGF), or a combination of any of the foregoing. In some embodiments, the EGF is human EGF, the FGF is human fibroblast growth factor 7 (FGF-7) or human fibroblast growth factor 10 (FGF-10), the HGF is human HGF, or the TGF is transforming growth factor-alpha (TGFα) (e.g., human TGFα). In some embodiments, the one or more receptor tyrosine kinase ligand includes human EGF, FGF-7, FGF-10, HGF, and TGFα.

In some embodiments of the foregoing aspect, one or more (e.g., two, three, four, or five) of the epithelial phenotype stabilizing agents is a transforming growth factor-beta (TGFβ) inhibitor. In some embodiments, the TGFβ inhibitor is an activin receptor-like kinase 5 (ALK5) inhibitor (e.g., A83-01). In some embodiments, one of the epithelial phenotype stabilizing agents is a corticosteroid (e.g., hydrocortisone).

In some embodiments of the foregoing aspect, the expansion medium further includes one or more cell survival agent and/or one or more cell proliferation agent. In some embodiments, the expansion medium includes a serum (e.g., fetal bovine serum). In some embodiments, the expansion medium does not include a serum (e.g., fetal bovine serum).

In some embodiments of the foregoing aspect, the expansion medium includes a serum replacement component. In some embodiments, the serum replacement component is KNOCKOUT™ Serum Replacement (KOSR), human platelet lysate, human serum, or bovine serum. In some embodiments, the serum replacement component v/v is 1% during culturing. In some embodiments, the serum replacement component v/v is increased in a gradient of Jan. 5, 2010% during culturing. In some embodiments, the serum replacement component v/v is increased in a gradient of 5-10% during culturing. In some embodiments, the expansion medium does not include a serum replacement component.

In some embodiments of the foregoing aspect, the expansion medium includes a Rho kinase inhibitor. In some embodiments, the Rho kinase inhibitor is Y-27632.

In some embodiments, the expansion medium does not include a Rho kinase inhibitor.

In some embodiments of the foregoing aspect, the ECM includes a collagen (e.g., collagen-I or collagen-IV) or a laminin (e.g., laminin-111, laminin-211, laminin-221, laminin-332, laminin-411, laminin-421, laminin-511, or laminin-521). In some embodiments, the ECM includes both a collagen and a laminin. In some embodiments, the ECM includes collagen-I. In some embodiments, the ECM includes collagen-IV. In some embodiments, the ECM includes laminin-111. In some embodiments, the ECM includes laminin-511. In some embodiments, the ECM includes laminin-521. In some embodiments, the ECM includes a combination of collagen-I, collagen-IV, laminin-111, laminin-511, and laminin-521. In some embodiments, the ECM does not include a hydrogel (e.g., MATRIGEL™). In some embodiments, the ECM includes a hydrogel (e.g., MATRIGEL™).

In some embodiments of the foregoing aspect, the culturing step is performed on a surface (e.g., a two-dimensional surface). In some embodiments, the surface is coated with the ECM. In some embodiments, the PHH are adherently attached to the surface during the culturing step. In some embodiments, expanded PHH are dissociated, aggregated, and maintained in culture to promote further expansion and/or maturation.

In some embodiments of the foregoing aspect, the expansion medium further includes a B27 supplement and/or an N2 supplement. In some embodiments, the B27 supplement does not contain vitamin A.

In some embodiments of the foregoing aspect, the expansion medium further includes an amino acid supplement. In some embodiments, the amino acid supplement is a non-essential amino acid (NEAA) supplement. In some embodiments, the NEAA supplement includes glycine, L-alanine, L-asparagine, L-aspartic acid, L-glutamic acid, L-proline, and L-serine. In some embodiments, the expansion medium does not include an amino acid supplement.

In some embodiments of the foregoing aspect, the expansion medium does not contain a Notch inhibitor or a Notch agonist. In some embodiments, the expansion medium does not contain gastrin.

In some embodiments, the expansion medium further includes a serum replacement component. In some embodiments, the serum replacement component includes KNOCKOUT™ Serum Replacement (KOSR), human platelet lysate, human serum, or bovine serum.

In some embodiments, the culturing includes culturing the cells under hypoxic conditions or in the presence of a hypoxia mimetic.

In some embodiments, the culturing is performed under hypoxic conditions. Hypoxic conditions may include, e.g., an oxygen level of less than 20%. In some embodiments, the culturing under hypoxic conditions includes culturing the cells at an oxygen level of between 1% to 19% (e.g., between 1% and 10%, e.g., 1%, 2%, 3%, 4%, 5%, 6%, 7%, 8%, 9%, 10%, 11%, 12%, 13%, 14%, 15%, 16%, 17%, 18%, 19%, or 20%). In some embodiments, the oxygen level is between 1% to 10%. In some embodiments, the oxygen level is 5%.

In some embodiments, the culturing step includes expanding plated cells (step P0) and a first passage of expanded cells (step P1). In some embodiments, the P0 step has a duration of between 7 to 16 days (e.g., 7, 8, 9, 10, 11, 12, 13, 14, 15, or 16 days). In some embodiments, the P0 step has a duration of 11 days. In some embodiments of the foregoing aspect, the P0 step has a duration of 13 days.

In some embodiments, the P1 step has a duration of between 7 to 20 days (e.g., 7, 8, 9, 10, 11, 12, 13, 14, 15, 16, 17, 18, 19, or 20 days). In some embodiments of the foregoing aspect, the P1 step has a duration of 11 days. In some embodiments, the P1 step has a duration of 13 days.

In some embodiments, the P0 step includes seeding the hepatocytes at a density of between 200 to 13,333 cells/cm², e.g., between 200 cells/cm² and 1,000 cells/cm² (e.g., 200 cells/cm², 300 cells/cm², 400 cells/cm², 500 cells/cm², 600 cells/cm², 700 cells/cm², 800 cells/cm², 900 cells/cm², or 1,000 cells/cm²), 1,000 cells/cm² to 10,000 cells/cm² (e.g., 1,000 cells/cm², 2,000 cells/cm², 3,000 cells/cm², 4,000 cells/cm², 5,000 cells/cm², 6,000 cells/cm², 7,000 cells/cm², 8,000 cells/cm², 9,000 cells/cm², or 10,000 cells/cm²), or 10,000 cells/cm² to 13,333 cells/cm² (e.g., 10,000 cells/cm², 11,000 cells/cm², 12,000 cells/cm², 13,000 cells/cm², or 13,333 cells/cm²). In some embodiments, the P0 step includes seeding the hepatocytes at a density of 667 cells/cm². In some embodiments, the P1 step includes seeding the hepatocytes at a density of between 333 to 13,333 cells/cm². In some embodiments, the P1 step includes seeding the hepatocytes at a density of 1,333 cells/cm².

In some embodiments, the expansion medium includes a serum replacement component, and the concentration of the serum replacement component is varied over the duration of the culturing step. In some embodiments, the concentration of the serum replacement component is 1% (v/v) on Day 0 of the P0 step.

In some embodiments, the concentration of the serum replacement is raised to 5% (v/V) (i) when the cell density reaches between 15% to 30% (e.g., 15%, 20%, 25%, or 30%) confluency or (ii) between Day 3 to Day 7 (e.g., Day 3, Day 4, Day 5, Day 6, or Day 7) of the P0 step. In some embodiments, the concentration of the serum replacement is raised to 5% (v/v) on Day 5 of the P0 step. In some embodiments, the concentration of the serum replacement component is 5% (v/v) on Day 0 of the P0 step. In some embodiments, the concentration of the serum replacement component is 5% (v/v) on Day 0 of the P0 and remains 5% (v/v) until the concentration of the serum replacement component is increased. In some embodiments, the concentration of the serum replacement is raised to 10% (v/v) (i) when the cell density reaches between 40% to 60% (e.g., 40%, 45%, 50%, 55%, or 60%) confluency or (ii) between Day 7 to Day 13 (e.g., Day 7, Day 8, Day 9, Day 10, Day 11, Day 12, or Day 13) of the P0 step. In some embodiments, the concentration of the serum replacement is raised to 10% (v/v) on Day 9 of the P0 step.

In some embodiments, the concentration of the serum replacement component is 1% (v/v) on Day 0 of the P1 step. In some embodiments, the concentration of the serum replacement is raised to 5% (v/v) (i) when the cell density reaches between 15% to 30% % (e.g., 15%, 20%, 25%, or 30%) confluency or (ii) between Day 3 to Day 7 (e.g., Day 3, Day 4, Day 5, Day 6, or Day 7) of the P1 step. In some embodiments, the concentration of the serum replacement is raised to 5% (v/v) on Day 5 of the P1 step.

In some embodiments, the concentration of the serum replacement component is 5% (v/v) on Day 0 of the P1 step. In some embodiments, the concentration of the serum replacement component is 5% (v/v) on Day 0 of the P1 step and remains 5% (v/v) until the concentration of the serum replacement component is increased.

In some embodiments, the concentration of the serum replacement is raised to 10% (v/v) (i) when the cell density reaches between 40% to 60% (e.g., 40%, 45%, 50%, 55%, or 60%) confluency or (ii) between Day 5 to Day 13 (e.g., Day 5, Day 6, Day 7, Day 8, Day 9, Day 10, Day 11, Day 12, or Day 13) of the P1 step. In some embodiments of the foregoing aspect, the concentration of the serum replacement is raised to 10% (v/v) on Day 7 of the P1 step. In some embodiments, the concentration of the serum replacement is raised to 10% (v/v) on Day 9 of the P1 step.

In some embodiments of the foregoing aspect, the method further includes determining, following the culturing step, the expression profile of the PHH.

In some embodiments of the foregoing aspect, the duration of the culturing step is for 3 days to 120 days (e.g., from about 15 days to about 120 days, from about 16 days to 119 days, from about 17 days to about 118 days, from about 18 days to about 117 days, from about 19 days to about 116 days, from about 20 days to about 115 days, from about 25 days to about 110 days, from about 30 days to about 100 days, from about 40 days to about 90 days, from about 50 days to about 80 days, from about 60 days to about 70 days, from about 65 days).

In some embodiments of the foregoing aspect, the culturing step is carried out using a multi-well plate or flask or container with a surface area of between 9.5 cm² to 10,000 cm². In some embodiments of the foregoing aspect, the culturing step is carried out using a multi-well plate or flask or container with a surface area of between 500 cm² to 10,000 cm². For example, in some embodiments, the surface area is between 9.5 cm² to 500 cm² (e.g., 9.5 cm², 100 cm², 200 cm², 300 cm², 400 cm², or 500 cm²), 500 cm² and 1,000 cm² (e.g., 500 cm², 600 cm², 700 cm², 800 cm², 900 cm², or 1,000 cm²), or 1,000 cm² to 10,000 cm² (e.g., 1,000 cm², 2,000 cm², 3,000 cm², 4,000 cm², 5,000 cm², 6,000 cm², 7,000 cm², 8,000 cm², 9,000 cm², or 10,000 cm²). In some embodiments, the culturing step is carried out using a flask or container with a surface area of between 636 cm² to 6360 cm².

In some embodiments of the foregoing aspect, following the culturing step, the expression profile of the PHH includes expression of one or more (e.g., two, three, four, or five) protein selected from hepatocyte nuclear factor 4 alpha (HNF4α), leucine rich repeat containing G protein-coupled receptor 5 (LGR5), keratin 18 (CK18), and albumin by at least 80% (e.g., at least 85%, 90%, or 95%) of the PHH.

In some embodiments of the foregoing aspect, following the culturing step, the expression profile of the PHH includes expression of Ki67 by at most 15% (e.g., at most 10%, 5%, 4%, 3%, 2%, or 1%) of the PHH.

In some embodiments of the foregoing aspect, following the culturing step, the expression profile of the PHH includes expression of Ki67 by at least 15% (e.g., at least 15%, 20%, 25%, 30%, 35%, 40%, 45%, 50%, 55%, 60%, 65%, 70%, 75%, or more) of the PHH.

In some embodiments of the foregoing aspect, following the culturing step, the expression profile of the PHH includes downregulation of one or more (e.g., two, three, four, or five) protein selected from mature hepatocyte markers (e.g., Cyp3a4, Cyp1a2, NR112, Urea cycle enzymes, ABCG2, ABCC2, ABCB11, SR-B1, or SLC10A1) by at least 10% (e.g., at least 11%, 12%, 13%, 14%, 15%, or 20%) of the overnight plated control PHH.

In some embodiments of the foregoing aspect, following the culturing step, the expression profile of the PHH includes upregulation of one or more (e.g., two, three, four, or five) proteins selected from fetal/hepatic progenitor/cholangiocyte markers (e.g., AFP, Cyp3a7, EPCAM, LGR5, KRT7, KRT19, or AQP1) by at least 10% (e.g., at least 11%, 12%, 13%, 14%, 15%, or 20%) of the overnight plated control PHH.

In some embodiments, expanded PHH need additional maturation step to upregulate urea cycle enzymes, proteins characteristic of mature cell state and downregulate fetal/hepatic progenitor/cholangiocyte markers.

In some embodiments of the foregoing aspect, following the culturing step, the expression profile of the PHH includes expression of one or more (e.g., two, three, four, or five) proteins selected from HNF4α, LGR5, CK18, and ALB by at least 80% (e.g., at least 85%, 90%, or 95%) of the PHH.

In some embodiments of the foregoing aspect, following the culturing step, the expression profile of the expanded PHH includes expression of Ki67 by at most 15% (e.g., at most 10%, 5%, 4%, 3%, 2% or 1%) of the PHH.

In some embodiments of the foregoing aspect, following the culturing step, the expression profile of the expanded PHH includes expression of Ki67 by at least 15% (e.g., at least 15%, 20%, 25%, 30%, 35%, 40%, 45%, 50%, 55%, 60%, 65%, 70%, 75%, or more) of the PHH.

In some embodiments of the foregoing aspect, following the culturing step, PHH continue to express and secrete albumin at levels lower than unexpanded PHH.

In some embodiments of the foregoing aspect, following the culturing step, the expression profile of the PHH includes increased expression of urea upon maturation of the PHHs.

In some embodiments of the foregoing aspect, following the culturing step, the hepatocyte yield is at least 5×10³ (e.g., at least 5×10³, 5×10⁴, 5×10⁵, 5×10⁶, or 5×10⁷) PHH per 1 cm².

In some embodiments of the foregoing aspect, following the culturing step, the hepatocyte yield expands by at least 2-fold (e.g., at least 3-fold, 4-fold, 5-fold, 10-fold, 20-fold, or 30-fold) within 14-28 days (e.g., within 15-27 days, within 16-26 days, within 17-25 days, within 18-24 days, within 19-23 days, within 20-21 days, or within 22 days) of culturing.

In some embodiments of the foregoing aspect, following the culturing step, the hepatocyte yield expands by at least 500-fold (e.g., at least 1000-fold) within 30 days (e.g., within 30, 29, 28, 27, 26, 25, 24, 23, 22, 21, 20, 19, 18, 17, 16, 15, or 14 days) of culturing. In some embodiments of the foregoing aspect, following the culturing step, the hepatocyte yield expands by at least 500-fold within 24 days of culturing.

In some embodiments of the foregoing aspect, following the culturing step, the hepatocyte yield expands by between 500-fold to 2000-fold, e.g., 1000-fold to 2000-fold (e.g., 500-fold, 600-fold, 700-fold, 800-fold, 900-fold, 1,000-fold, 1,100-fold, 1,200-fold, 1,300-fold, 1,400-fold, 1,500-fold, 1,600-fold, 1,700-fold, 1,800-fold, 1,900-fold, or 2,000-fold) within 30 days (e.g., within 30, 29, 28, 27, 26, 25, 24, 23, 22, 21, 20, 19, 18, 17, 16, 15, or 14 days) of culturing. In some embodiments of the foregoing aspect, following the culturing step, the hepatocyte yield expands by between 500-fold to 2000-fold, e.g., 1000-fold to 2000-fold (e.g., 500-fold, 600-fold, 700-fold, 800-fold, 900-fold, 1,000-fold, 1,100-fold, 1,200-fold, 1,300-fold, 1,400-fold, 1,500-fold, 1,600-fold, 1,700-fold, 1,800-fold, 1,900-fold, or 2,000-fold) within 24 days of culturing.

In some embodiments of any of the foregoing aspects, the method further includes maturing the hepatocytes in a maturation medium including a basal medium for human cells to which is added one or more hepatocyte maturation supplements.

In another aspect, the disclosure provides a method for maturing a population of hepatocytes, which includes the step of maturing an expanded hepatocyte population in the presence of a maturation medium including a basal medium for human cells to which is added one or more hepatocyte maturation supplements. In some embodiments of the foregoing aspect, prior to maturing, the hepatocytes were expanded by culturing the hepatocytes in contact with an ECM in the presence of an expansion medium including a basal medium, a Wnt signaling activator, one or more receptor tyrosine kinase ligand, and one or more epithelial phenotype stabilizing agent. In some embodiments of the foregoing aspect, the expanded hepatocytes have an immature phenotype.

In some embodiments of any of the foregoing aspects, the maturation step begins immediately following hepatocyte expansion. In some embodiments of any of the foregoing aspects, the maturation step does not begin immediately following hepatocyte expansion. In some embodiments of any of the foregoing aspects, the maturation step has a duration of between 3 and 12 days (e.g., 3, 4, 5, 6, 7, 8, 9, 10, 11, or 12 days). In some embodiments of any of the foregoing aspects, the maturation step is 7 days.

In some embodiments of any of the foregoing aspects, the maturation basal medium is LONZA™ HCM™, William's E, or HepatoZYME-SFM. In some embodiments of any of the foregoing aspects, the one or more maturation supplements includes an antibiotic, HEPES, GLUTAMAX™, ITS, a Notch inhibitor, an EGFR inhibitor, Oncostatin M, an anti-oxidant, a glucocorticoid, a pregnane X receptor (PXR) activator, a bile acid, a CAMP or cAMP analog, cholesterol, a thyroid hormone, a serum replacement component, or any combination of the foregoing.

In some embodiments of the foregoing aspect, the Notch inhibitor is Compound E, Gamma Secretase Inhibitor XX, or a combination thereof. In some embodiments of the foregoing aspect, the antibiotic is penicillin, streptomycin, or a combination thereof. In some embodiments of the foregoing aspect, the EGFR inhibitor is erlotinib HCl. In some embodiments of the foregoing aspect, the anti-oxidant is vitamin C. In some embodiments of the foregoing aspect, the glucocorticoid is dexamethasone, hydrocortisone, or a combination thereof. In some embodiments of the foregoing aspect, the PXR activator is vitamin K2. In some embodiments of the foregoing aspect, the bile acid is lithocholic acid, urso deoxycholic acid, or a combination thereof. In some embodiments of the foregoing aspect, the CAMP analog is 8 bromo CAMP, forskolin, or a combination thereof. In some embodiments of the foregoing aspect, the thyroid hormone is T3. In some embodiments of the foregoing aspect, the serum replacement component is Insulin Transferrin Selenium (ITS), KOSR, Trace Elements A, Trace Elements B, or a combination thereof.

In some embodiments of any of the foregoing aspects, the maturation medium lacks one or more of R-spondin 1/Wnt3a, Epidermal Growth Factor (EGF), Transforming Growth Factor α (TGFα), N-acetyl cysteine, Nicotinamide, B27 supplement, N2 supplement, Fibroblast Growth Factor 7 (FGF7), and Fibroblast Growth Factor 10 (FGF10).

In another aspect, the disclosure provides a kit including an expansion medium including a basal medium for human cells to which is added one or more Wnt signaling activator, one or more receptor tyrosine kinase ligand, and one or more epithelial phenotype stabilizing agent, wherein the kit further includes a package insert instructing a user of the kit to culture one or more hepatocyte in accordance with the method of any one of the foregoing embodiments.

In another aspect, the disclosure provides an expansion medium including a basal medium for human cells to which is added one or more (e.g., two, three, four, or five) Wnt signaling activators, one or more (e.g., two, three, four, or five) receptor tyrosine kinase ligand, and one or more (e.g., two, three, four, or five) epithelial phenotype stabilizing agent.

In some embodiments of any of the foregoing aspects, one or more (e.g., two, three, four, or five) of the Wnt signaling activators is R-spondin 1, R-spondin 2, R-spondin 3, R-spondin 4, Wnt3a, or a combination of any of the foregoing. In some embodiments, the one or more Wnt signaling activator includes R-spondin 1 and Wnt3a.

In some embodiments of any of the foregoing aspects, one or more (e.g., two, three, four, or five) of the receptor tyrosine kinase ligands is an epidermal growth factor (EGF), a fibroblast growth factor (FGF), a hepatocyte growth factor (HGF), a transforming growth factor (TGF), or a combination of any of the foregoing. In some embodiments, the EGF is human EGF, the FGF is human fibroblast growth factor 7 (FGF-7) or human fibroblast growth factor 10 (FGF-10), the HGF is human HGF, or the TGF is transforming growth factor-alpha (TGFα) (e.g., human TGFα). In some embodiments, the one or more receptor tyrosine kinase ligand includes human EGF, FGF-7, FGF-10, HGF, and TGFα.

In some embodiments of the foregoing aspect, the expansion medium includes a Rho kinase inhibitor. In some embodiments, the Rho kinase inhibitor is Y-27632.

In some embodiments, the expansion medium does not include a Rho kinase inhibitor.

In some embodiments of any of the foregoing aspects, one or more (e.g., two, three, four, or five) of the epithelial phenotype stabilizing agents is a transforming growth factor-beta (TGFβ) inhibitor. In some embodiments, the TGFβ inhibitor is an activin receptor-like kinase 5 (ALK5) inhibitor (e.g., A83-01). In some embodiments, one of the epithelial phenotype stabilizing agents is a corticosteroid (e.g., hydrocortisone).

In some embodiments of any of the foregoing aspects, the expansion medium further includes one or more cell survival agent and/or one or more cell proliferation agent.

In some embodiments of any of the foregoing aspects, the ECM includes a collagen (e.g., collagen-I or collagen-IV) or a laminin (e.g., laminin-111, laminin-211, laminin-221, laminin-332, laminin-411, laminin-421, laminin-511, or laminin-521). In some embodiments, the ECM includes collagen-I. In some embodiments, the ECM includes collagen-IV. In some embodiments, the ECM includes laminin-111. In some embodiments, the ECM includes laminin-511. In some embodiments, the ECM includes laminin-521. In some embodiments, the ECM includes a combination of collagen-I, collagen-IV, laminin-111, laminin-511, and laminin-521.

In some embodiments of any of the foregoing aspects, the culturing step is performed on a surface (e.g., a two-dimensional surface). In some embodiments, the surface is coated with the ECM. In some embodiments, the PHH are adherently attached to the surface during the culturing step. In some embodiments, expanded PHHs are dissociated, aggregated, and maintained in culture to promote further expansion and/or maturation.

In some embodiments of any of the foregoing aspects, the expansion medium further includes a B27 supplement and/or an N2 supplement. In some embodiments, the B27 supplement does not contain vitamin A.

In some embodiments of any of the foregoing aspects, the expansion medium does not contain a Notch inhibitor or a Notch agonist. In some embodiments, the expansion medium does not contain gastrin.

In some embodiments of the foregoing aspect, the expansion medium further includes an amino acid supplement. In some embodiments, the amino acid supplement is a non-essential amino acid (NEAA) supplement. In some embodiments, the NEAA supplement includes glycine, L-alanine, L-asparagine, L-aspartic acid, L-glutamic acid, L-proline, and L-serine. In some embodiments, the expansion medium does not include an amino acid supplement.

In another aspect, the disclosure provides a tissue culture vessel that includes an expansion medium as described herein (e.g., of any of the above embodiments).

In another aspect, the disclosure provides an incubator that maintains the tissue culture vessel under hypoxic conditions. Hypoxic conditions may include, e.g., an oxygen level of less than 20%. In some embodiments, the incubator maintains an oxygen level of between 1% to 19% (e.g., between 1% and 10%, e.g., 1%, 2%, 3%, 4%, 5%, 6%, 7%, 8%, 9%, 10%, 11%, 12%, 13%, 14%, 15%, 16%, 17%, 18%, 19%, or 20%). In some embodiments, the oxygen level is between 1% to 10%. In some embodiments, the oxygen level is 5%.

In another aspect, the disclosure provides a kit including a maturation medium including a basal medium for human cells to which is added one or more maturation supplements, wherein the kit further includes a package insert instructing a user of the kit to mature one or more hepatocyte in accordance with the method of any one of the foregoing embodiments.

In another aspect, the disclosure provides a maturation medium including a basal medium for human cells to which is added one or more hepatocyte maturation supplements.

In some embodiments of the foregoing aspect, the maturation basal medium is LONZA™ HCM™, William's E, or HepatoZYME-SFM. In some embodiments of the foregoing aspect, the one or more maturation supplements includes an antibiotic, 4-(2-hydroxyethyl)-1-piperazineethanesulfonic acid (HEPES), GLUTAMAX™, ITS, a Notch inhibitor, an EGFR inhibitor, Oncostatin M, an anti-oxidant, a glucocorticoid, a pregnane X receptor (PXR) activator, a bile acid, a cAMP or cAMP analog, cholesterol, a thyroid hormone, a serum replacement component, or any combination of the foregoing.

In some embodiments of the foregoing aspect, the Notch inhibitor is Compound E, Gamma Secretase Inhibitor XX, or a combination thereof. In some embodiments of the foregoing aspect, the antibiotic is penicillin, streptomycin, or a combination thereof. In some embodiments of the foregoing aspect, the EGFR inhibitor is erlotinib HCl. In some embodiments of the foregoing aspect, the anti-oxidant is vitamin C. In some embodiments of the foregoing aspect, the glucocorticoid is dexamethasone, hydrocortisone, or a combination thereof. In some embodiments of the foregoing aspect, the PXR activator is vitamin K2. In some embodiments of the foregoing aspect, the bile acid is lithocholic acid, urso deoxycholic acid, or a combination thereof. In some embodiments of the foregoing aspect, the CAMP analog is 8 bromo CAMP, forskolin, or a combination thereof. In some embodiments of the foregoing aspect, the thyroid hormone is T3. In some embodiments of the foregoing aspect, the serum replacement component is ITS, KOSR, Trace Elements A, Trace Elements B, or a combination thereof.

In some embodiments of any of the foregoing aspects, the maturation medium lacks one or more of R-spondin 1/Wnt3a, Epidermal Growth Factor (EGF), Transforming Growth Factor α (TGFα), N-acetyl cysteine, Nicotinamide, B27 supplement, N2 supplement, Fibroblast Growth Factor 7 (FGF7), and Fibroblast Growth Factor 10 (FGF10).

### BRIEF DESCRIPTION OF THE DRAWINGS

FIG. 1 is a brightfield image of primary human hepatocyte (PHH) cells cultured using an expansion medium referred to as Expand 1.0 formulation, which included advanced Dulbecco's Modified Eagle Medium/Ham's Nutrient Mixture F-12, to which was added the Wnt signaling activators R-spondin 1 and Wnt3a; the receptor tyrosine kinase ligands recombinant epidermal growth factor, recombinant transforming growth factor-alpha, recombinant human fibroblast growth factor 7, recombinant human fibroblast growth factor 10, and recombinant human hepatocyte growth factor; the epithelial phenotype stabilizing agent A83-01; N-acetylcysteine, nicotinamide, the Rho kinase inhibitor Y-27632; a B27 supplement that did not contain vitamin A and a N2 supplement; -2-hydroxyethylpiperazine-N′-2-ethanesulfonic acid (HEPES), GLUTAMAX™, penicillin, and streptomycin. PHH cells are shown after 6 days of plating and culturing (passage “P” 0) in a T-75 culture flask.

FIG. 2 is a set of photomicrographs showing brightfield images of cells on Day 2, Day 9 and Day 14 of expansion and immunostaining of markers on Day 13 of expansion for hepatic phenotype in PHH cultured in expansion medium using a hepatocyte lot (lot D in Table 3) that expands as a homogenous culture of hepatic lineage cells. PHH were cultured on Collagen-I extracellular matrix (ECM) in the presence of an expansion medium. The expansion medium was as described in FIG. 1. At day 13 of culture, PHH express HNF4α, Albumin, and LGR5.

FIG. 3 is a set of photomicrographs showing brightfield images of cells on Day 2, Day 10, and Day 13 during expansion and immunostaining of markers on Day 13 of expansion for hepatic phenotype in PHH expanded on Laminin-521 ECM using the expansion medium described in FIG. 1 using a hepatocyte lot described in FIG. 2. At day 13 of P0 culture, PHH express HNF4α, Albumin, and LGR5.

FIG. 4 is a set of photomicrographs showing brightfield images of expanded PHH cultured in the presence of the expansion medium described in FIG. 1 using a hepatocyte lot (lot A in Table 3) that expands as a heterogenous culture consisting of hepatic islands in the background of mesenchymal-like cells at day 20 (passage (“P”) 0), day 76 (P3), day 89 (P4), and day 113 (P5), respectively.

FIG. 5 is a set of photomicrographs showing immunostaining of markers for hepatic phenotype and a proliferative phenotype in PHH cultured in the presence of the expansion medium described in FIG. 1. Images depict the leading edge of colonies staining positive for selected markers during extended culture of a PHH lot described in FIG. 4. At day 68, PHH express HNF4a, Albumin, LGR5, and Ki67.

FIG. 6 is a set of photomicrographs showing the stages of expanding PHH cultured in the presence of the expansion medium described in FIG. 1 of a PHH lot described in FIG. 2. Cells progressively expand in culture and reach confluence at day 9 of P0, further progressing to become tightly packed with well-defined cell margins by day 17 of P0.

FIG. 7 is a graph showing the quantification of fold expansion after 17 days of culture in P0 based on number of PHH cells seeded and cultured in the presence of the expansion medium described in FIG. 1 of a PHH lot described in FIG. 2.

FIG. 8 is a graph showing secreted human albumin in NSG mice transplanted with grafts containing expanded PHH of a hepatocyte lot described in FIG. 2. Unexpanded PHH served as reference control group. Unexpanded PHH and PHH cultured in the presence of the expansion medium described in FIG. 1 for 10 days were dissociated, aggregated with fibroblasts, and grafts prepared by encapsulating in fibrin hydrogel followed by transplantation into NSG mice. Blood was drawn every two weeks and secreted human albumin was quantified by ELISA. Human albumin is shown at Days 4, 8, 13, 18, 22, 27, 32, 36, 41, 46, 50, 55, and 61.

FIG. 9 is a set of photomicrographs showing immunohistochemistry of grafts explanted on day 61 containing unexpanded control PHH and PHH cultured for 10 days in the presence of the expansion medium described in FIG. 1. H&E, hOTC, and Ck18 staining are shown.

FIG. 10 is a set of photomicrographs showing brightfield images of expanding cells on different days of PHH cultured in the presence of a modified expansion medium referred to as Expand 3.0 formulation in hypoxic culture condition (5% O₂) on Collagen-I and Laminin-521 ECM in P0 and P1. Modified expansion medium included advanced Dulbecco's Modified Eagle Medium/Ham's Nutrient Mixture F-12, to which was added the Wnt signaling activators R-spondin 1 and Wnt3a; the receptor tyrosine kinase ligands recombinant epidermal growth factor, recombinant transforming growth factor-alpha, recombinant human fibroblast growth factor 7, recombinant human fibroblast growth factor 10, and recombinant human hepatocyte growth factor; the epithelial phenotype stabilizing agent A83-01; N-acetylcysteine, nicotinamide; 5-10% KNOCKOUT™ serum replacement (KOSR); non-essential amino acids (NEAAs); a B27 supplement that did not contain vitamin A and a N2 supplement; -2-hydroxyethylpiperazine-N′-2-ethanesulfonic acid (HEPES), GLUTAMAX™, penicillin, and streptomycin.

FIG. 11 is a graph comparing the cumulative fold expansion (P0+P1) of PHH cultured in a modified expansion medium described in FIG. 10 on Collagen-I and Laminin-521 ECM under hypoxic culture condition (5% O₂).

FIG. 12 is a heat map of RT-qPCR analysis using TaqMan probes covering different categories involving mature and progenitor cell state of hepatocytes and epithelial and mesenchymal markers on overnight plated control PHH cells in the absence of expansion medium and PHH cultured in the presence of Expand 3.0 formulation in hypoxic culture condition (5% O₂) in P0 and P1 on Collagen-I and Laminin-521 ECM. Values are normalized against sample #1 of overnight plated control hepatocytes on Collagen-I matrix. LGR5 data is presented as a separate bar graph for expanded cells only as LGR5 is not detected in overnight plated control hepatocytes.

FIG. 13 is a graph showing the level of secreted albumin by overnight plated control PHH cells in the absence of expansion medium, as compared to PHH cultured in the presence of Expand 3.0 formulation in hypoxic culture condition (5% O₂) in P0 and P1 on Collagen-I and Laminin-521 ECM.

FIG. 14 is a set of photomicrographs showing immunostaining of markers for hepatic phenotype in PHH cultured in the presence of Expand 3.0 formulation in hypoxic culture condition on Laminin-521 ECM for 24 days (13 in P0+11 days in P1). At day 11 of P1 culture, PHH express HNF4a and Albumin.

FIG. 15 is a table and graph showing percentage of cells at day 11 of P1 culture expressing HNF4a and Albumin from two independent wells of a 6-well plate as determined by Imaris software.

FIG. 16 is a diagram depicting a timeline of expansion and maturation of PHH alongside of the KOSR percentage during passage (p) on various days (d). Phase contrast microscopy, secreted urea, secreted albumin, and RT-qPCR of maturation markers were used for readouts of cell maturation.

FIG. 17 is a graph showing secreted urea in μg/million cells/day for primary hepatocytes (Prim Hep), expanded hepatocytes (Expanded Hep control), and expanded matured hepatocytes in either Lonza HCM medium (Mat Suppl in Lonza HCM) or William's E medium (Mat Suppl in William's E). Primary hepatocytes are shown normalized to cells that were plated down and normalized to total cells that were seeded. Error bars=SEM with n=2 for maturation conditions and n=3 for primary hepatocyte and expanded hepatocyte controls.

FIG. 18 is a graph showing secreted albumin in ng/million cells/day for primary hepatocytes (Prim Hep), expanded hepatocytes (Expanded Hep control), and expanded matured hepatocytes in either Lonza HCM medium (Mat Suppl in Lonza HCM) or William's E medium (Mat Suppl in William's E). Primary hepatocytes are shown normalized to cells that were plated down and normalized to total cells that were seeded. Error bars=SEM with n=2 for maturation conditions and n=3 for primary hepatocyte and expanded hepatocyte controls.

FIG. 19 is a heat map showing the fold change in transcript levels measured using RT-PCR of mature hepatocyte markers and progenitor/cholangiocyte markers for primary hepatocytes (Prim Hep Control), expanded hepatocytes (Expanded Hep control), and expanded matured hepatocytes in either Lonza HCM medium (Mat Suppl in Lonza HCM) or William's E medium (Mat Suppl in William's E).

FIG. 20 is a collection of phase contrast microscopy images showing hepatocytes before, during, and after completion of maturation. Expanded hepatocytes are shown at 10× magnification on Day 24 before starting maturation. Mature expanded hepatocytes (Mat Suppl in Lonza HCM) are shown at 4× and 10× magnification on Day 1, Day 3, and Day 7 of maturation. Scale bar=100 microns.

### DEFINITIONS

As used herein, the terms “a,” “an,” and “the” include plural referents unless the content clearly dictates otherwise. Thus, for example, reference to “a ligand” optionally includes a combination of two or more such ligands, and the like.

As used herein, the term “basal medium” refers to any cell culture medium which is appropriate for culturing human cells, including, but not limited to, Dulbecco's Modified Eagle Medium/Ham's Nutrient Mixture F-12 (DMEM/F-12), LONZA™ HCM™, William's E, HepatoZYME-SFM and any modification thereof. Modifications to the cell culture medium include various reagent additions including, but not limited to, stable forms of L-glutamine, buffering agents such as -2-hydroxyethylpiperazine-N′-2-ethanesulfonic acid (HEPES), and antibiotic solutions, such as penicillin and streptomycin.

As used herein, the terms “comprise,” “comprising,” “comprises,” and “comprised of” are synonymous with “include,” “including,” “includes,” or “contain,” “containing,” “contains,” and are inclusive or open-ended terms that specify the presence of what follows, e.g., a component, and do not exclude or preclude the presence of additional, non-recited components, features, element, members, steps, known in the art or disclosed therein.

As used herein, the term “control cells” refers to a population of hepatocytes cultured in a control medium including any appropriate basal medium for human cells (e.g., Dulbecco's Modified Eagle Medium/Nutrient Mixture F-12) supplemented with mammalian serum (e.g., fetal bovine serum) and any other vendor-provided media supplements appropriate for hepatocytes.

As used herein, the term “culturing step” refers to the process of expanding and passaging cells. This process encompasses the phase of cell culture in which the number of cells increases by cell division. When the cells have reached, e.g., 80-90% confluence, they may be passaged and seeded onto additional cell culture surfaces. For example, one passage of primary human hepatocytes (PHH) may require at least 3 days to reach confluence. Cells can be continuously passaged and cultured for 120 days. Alternatively, cells can be passaged until they become transformed or lose hepatic phenotype.

The terms “decrease,” “reduced,” “reduction,” or “inhibit” are all used herein to mean a decrease by a statistically significant amount. In some embodiments, “reduce,” “reduction,” “decrease,” or “inhibit” typically means a decrease by at least 10% as compared to a reference level (e.g., the absence of a given treatment) and can include, for example, a decrease by at least about 10%, at least about 20%, at least about 30%, at least about 35%, at least about 40%, at least about 45%, at least about 50% at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” may encompass a complete inhibition or reduction as compared to a reference level.

As used herein, the term “epithelial phenotype stabilizing agent” refers to any compound, natural or synthetic, that can promote maintenance of the epithelial phenotype. An epithelial phenotype stabilizing agent may also prevent development of the mesenchymal phenotype. Such compounds include, but are not limited to, inhibitors of the TGFβ signaling pathway and corticosteroids.

As used herein, the terms “expand,” “expands,” “expanding,” and “expansion” refer to an increase in the number of what follows, e.g., a population of PHH. An “expansion step” refers to a phase of cell culture in which the number of PHH increases by cell division. As used herein in the context of a plurality of agents that together or collectively “expand” a population of PHH, describes instances in which each agent, individually, may or may not achieve the indicated function, but when the agents are combined, the indicated expansion is achieved.

As used herein, the term “express” refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein. In the context of a gene that encodes a protein product, the terms “gene expression” and the like are used interchangeably with the terms “protein expression” and the like. Expression of a gene or protein of interest in a patient can manifest, for example, by detecting: an increase in the quantity or concentration of mRNA encoding corresponding protein (as assessed, e.g., using RNA detection procedures described herein or known in the art, such as reverse transcription quantitative polymerase chain reaction (RT-qPCR) and RNA seq techniques), an increase in the quantity or concentration of the corresponding protein (as assessed, e.g., using protein detection methods described herein or known in the art, such as enzyme-linked immunosorbent assays (ELISA), among others), and/or an increase in the activity of the corresponding protein (e.g., in the case of an enzyme, as assessed using an enzymatic activity assay described herein or known in the art) in a sample obtained from the patient. As used herein, a cell is considered to “express” a gene or protein of interest if one or more, or all, of the above events can be detected in the cell or in a medium in which the cell resides. For example, a gene or protein of interest is considered to be “expressed” by a cell or population of cells if one can detect (i) production of a corresponding RNA transcript, such as an mRNA template, by the cell or population of cells (e.g., using RNA detection procedures described herein); (ii) processing of the RNA transcript (e.g., splicing, editing, 5′ cap formation, and/or 3′ end processing, such as using RNA detection procedures described herein); (iii) translation of the RNA template into a protein product (e.g., using protein detection procedures described herein); and/or (iv) post-translational modification of the protein product (e.g., using protein detection procedures described herein).

The terms “expression level” or “level of expression” in general are used interchangeably and generally refer to the amount of a marker in a biological sample. “Expression” generally refers to the process by which information (e.g., gene-encoded and/or epigenetic information) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., post-translational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs).

As used herein, the term “hypoxic conditions” refers to conditions in which the atmospheric oxygen level is below 20.9%.

The terms “increased,” “increase,” “enhance,” or “activate” are all used herein to mean an increase by a statistically significant amount. In some embodiments, the terms “increased,” “increase,” “enhance,” or “activate” can mean an increase of at least 10% as compared to a reference level, or at least about a 2-fold, or at least about a 3-fold, or at least about a 4-fold, or at least about a 5-fold, or at least about a 10-fold increase, or any increase between 2-fold and 10-fold or greater as compared to a reference level. In the context of a marker (e.g., albumin), an “increase” is a statistically significant increase in such level.

As used herein, the term “inhibitor” refers to any compound, natural or synthetic, which can reduce the activity of a target protein or signaling pathway. An inhibitor may attenuate or prevent the activity of a target protein either directly or indirectly. Direct inhibition can be obtained, for instance, by binding to a protein and preventing the protein from interacting with an endogenous molecule, such as an enzyme, a substrate, or other binding partner, thereby diminishing the activity of the protein. For instance, an inhibitor may bind an enzyme active site and sterically preclude binding of an endogenous substrate at this location, thus decreasing the enzymatic activity of the protein. Alternatively, indirect inhibition can be obtained, for instance, by binding to a protein that promotes the activity of a target protein by inducing a conformational change or catalyzing a chemical modification of the target protein. For instance, indirect inhibition of a target protein may be achieved by binding and inactivating a kinase that catalyzes the phosphorylation of, and thus activates, the target protein.

As used herein, the term “level” refers to a level of a protein, as compared to a reference. The reference can be any useful reference, as defined herein. By a “decreased level” and an “increased level” of a protein is meant a decrease or increase in protein level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an 15 increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). A level of a protein may be expressed in mass/vol (e.g., g/dL, mg/mL, μg/mL, or ng/mL) or percentage relative to total protein in a sample.

As used herein, the term “marker” is used interchangeably herein to refer to a DNA, RNA, protein, carbohydrate, or glycolipid-based molecular marker, the expression or presence of which in a cell sample can be detected by standard methods (or methods disclosed herein). Expression of such a marker may be determined to be higher or lower in a population of PHH expanded and/or matured using the disclosed compositions and/or according to the disclosed methods, as compared to a population of unexpanded and/or unmatured PHH not treated with said composition or methods.

As used herein, the term “maturing” refers to the process of developing cells into a population that is distinct from a progenitor population. For example, in some instances, a population of hepatocytes that has undergone a maturing step as disclosed herein has a mature hepatocyte phenotype (e.g., as assessed by gene expression analysis (e.g., an upregulation of transcripts associated with mature hepatocytes and downregulation of transcripts associated with progenitors/cholangiocytes), hepatocyte functional assays (e.g., urea secretion, CYP3A4 activity).

As used herein, the terms “one or more” or “at least one,” such as one or more or at least one member(s) of a group of members, is clear per se, by means of further exemplification, the term encompasses inter alia a reference to any one of said members, or to any two or more of said members, e.g., ≥3, ≥4, ≥5, ≥6, or ≥7, etc., of said members, and up to all said members.

As used herein, the term “pharmaceutical formulation” represents a composition containing a population of primary human hepatocytes (PHH) expanded and/or matured according to the methods described herein, formulated with a pharmaceutically acceptable excipient, and manufactured or sold with the approval of a governmental regulatory agency as part of a therapeutic regimen for the treatment of disease in a subject.

“Primary cells,” and “primary cultures” are used interchangeably herein to refer to cells and cell cultures that have been harvested from human livers and cryogenically stored immediately without being cultured in vitro for any amount of time. Cells can be harvested from an individual by any convenient method such as biopsy or isolation from whole donated livers. An appropriate solution can be used for dispersion or suspension of the harvested cells. The cells can be used immediately, or they can be stored, frozen, for long periods of time, being thawed and capable of being reused. In such cases, the cells will usually be frozen in 10% DMSO, 50% serum, 40% buffered medium, or some other solution as is commonly used in the art to preserve cells at such freezing temperatures and thawed in a manner as commonly known in the art for thawing frozen cultured cells.

As used herein, the term “primary human hepatocytes” (or “PHH”) refer to major parenchymal cells in the liver. Specifically, these cells are of human origin and have the capacity to replicate and increase cell number in response to liver injury. It is known in the art that such cells express one or more gene selected from HNF4α, albumin, and a member of the CYP gene family. It is also known that such cells do not express the AFP gene. It is additionally known in the art that such cells express proteins including, but not limited to, HNF4α, albumin, A1AT, Transferrin, and urea.

As used herein, the term “receptor tyrosine kinase ligand” refers to any compound native or recombinant, which can bind to a receptor tyrosine kinase. Such ligands include, but are not limited to, native and recombinant epidermal growth factor (EGF), native and recombinant fibroblast growth factor (FGF), native and recombinant hepatocyte growth factor (HGF), and native and recombinant transforming growth factor (TGF).

As used herein, the term “recipient” refers to any organism to which a composition in accordance with the invention may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition. In preferred embodiments, the subject is a human.

As used herein, the term “serum replacement component” refers to a component is present in a serum-free medium.

As used herein, a transforming growth factor beta “(TGFβ) inhibitor” refers to a substance (e.g., a small molecule, protein, interfering RNA, or other natural or synthetic compound) that can attenuate or prevent the transcription of one or more genes that are transcribed due to the activity of a SMAD transcription co-activator protein. A TGFβ inhibitor may disrupt the signal transduction cascade that leads to SMAD-induced gene transcription at one or more points within the pathway. For instance, a TGFβ inhibitor may disrupt or prevent TGFβ or a TGFβ superfamily ligand, such as activin, inhibin, nodal, lefty, bone morphogenetic protein (BMP), growth and differentiation factor (GDF), or mullerian inhibitory factor (MIF), from binding to its endogenous receptor, thus inhibiting the phosphorylation and activation of the receptor-associated SMAD proteins. A TGFβ pathway inhibitor may function by preventing the translocation of one or more SMAD proteins to the nucleus, for example, by binding a SMAD protein and preventing or disrupting the interaction between the SMAD protein and the nucleoporins. A TGFβ signaling pathway inhibitor may stabilize the interaction between one or more SMAD proteins and SMAD anchor for receptor activation (SARA), which sequesters SMAD proteins in the cytoplasm and prevents their translocation into the nucleus. Other examples of TGFβ signaling pathway inhibitors include substances, such as neurogenin, that bind SMAD proteins and sequester them from DNA-bound transcription factors, thus preventing transcription of a target gene. Alternative inhibitors of the TGFβ signaling pathway include substances that promote the ubiquitination of one or more SMAD proteins, thereby marking the protein for degradation by the proteasome and preventing target gene transcription. Exemplary assays that can be used to determine the inhibitory activity of a TGFβ signaling pathway inhibitor include, without limitation, electrophoretic mobility shift assays, antibody supershift assays, as well as TGFβ-inducible gene reporter assays, among others.

As used herein, a “Wnt signaling activator” refers to an agonist of the canonical Wnt signaling pathway. Agonists of this pathway further include Wnt proteins or other compounds that bind directly to the Frizzled and LRP56 co-receptor proteins in a manner that promotes an increase in the concentration of β-catenin in the nucleus of a mammalian cell. Wnt signaling activators include, but are not limited to, Wnt-3a (R&D systems). Alternatively, a Wnt pathway agonist may function by inhibiting one or more secreted Frizzled-related proteins (SFRPs) or Wnt inhibitory protein (WIF), which bind and sequester Wnt proteins from the endogenous Wnt co-receptors. These Wnt signaling activators can include molecules that activate the non-canonical Wnt signaling pathway by stimulating Wnt signaling in a Frizzled-independent manner. Non-canonical Wnt signaling activators preferably stimulate the Wnt pathway via the LGR5 cell surface receptor. Known LGR5 agonists include, but are not limited to, roof plate-specific spondin (R-spondin) proteins.

### DETAILED DESCRIPTION

The present disclosure provides compositions and methods that can be used for the two-dimensional expansion and three-dimensional aggregation of primary human hepatocytes (PHH). In accordance with the compositions and methods described herein, a recipient (e.g., a human) may be implanted with a population of the expanded PHH. The disclosed methods for expanding PHH result in robust and widely applicable culture expansion. Furthermore, these methods are not limited by the restrictive age limits on donor PHH, thereby expanding the pool of eligible PHH donors and feasibly reducing the shortage of donor PHH.

This invention is based, at least in part, on the discovery of completely defined culture conditions, including a basal medium for human cells, one or more Wnt signaling activators, one or more receptor tyrosine kinase ligands, one or more epithelial phenotype stabilizing agents, and optionally one or more cell survival agents or one or more cell proliferation agents, that allow PHH to be cultured for an increased number of passages as compared to existing cell culture medium formulations. The defined culture conditions may include the presence of a serum replacement component (e.g., KNOCKOUT™ Serum Replacement (KOSR)) and/or culturing under hypoxic conditions. The completely defined, xeno-free culture conditions also result in large-scale, robust, two-dimensional expansion of PHH that advantageously allows more cells to be obtained from a single starting cell or from a collection of starting cells than was not possible using previous methods. These advantages allow for increasing the availability of donor cells for therapeutic purposes (e.g., to supplement or rescue native liver function).

The present disclosure also provides compositions and methods that can be used for the maturation of PHH. In accordance with the compositions and methods described herein, a recipient (e.g., a human) may be implanted with a population of the matured hepatocytes. The disclosed methods for maturing PHH result in robust and widely applicable cell maturation.

This invention is based, at least in part, on the discovery of completely defined culture conditions for maturing PHH, including a basal medium for human cells with one or more hepatocyte maturation supplements, and optionally removing one or more supplements to prevent cell proliferation and/or the progenitor phenotype, that allow the hepatocytes to be matured into a distinct population.

In some embodiments, the method includes culturing one or more PHH in contact with an extracellular matrix (ECM) in the presence of an expansion medium including a basal medium for human cells to which is added one or more Wnt signaling activator, one or more receptor tyrosine kinase ligand, and one or more epithelial stabilizing agent.

In some embodiments, the expansion medium includes a basal medium for human cells to which is added one or more Wnt signaling activator, one or more receptor tyrosine kinase ligand, one or more epithelial stabilizing agent, and one or more cell survival agent.

In some embodiments, the expansion medium includes a basal medium for human cells to which is added one or more Wnt signaling activator, one or more receptor tyrosine kinase ligand, one or more epithelial stabilizing agent, and one or more cell proliferation agent.

In some embodiments, the expansion medium includes a basal medium for human cells to which is added one or more Wnt signaling activator, one or more receptor tyrosine kinase ligand, one or more epithelial stabilizing agent, one or more cell survival agent, and one or more cell survival agent.

In some embodiments, the maturation medium includes a basal medium for human cells to which is added one or more maturation supplements.

In some embodiments, the maturation medium does not include one or more supplements that promote cell proliferation. For example, compared to an expansion medium, a maturation medium disclosed herein may lack one or more supplements that promotes cell proliferation.

In some embodiments, the maturation medium does not include one or more supplements that promote a progenitor phenotype. For example, compared to an expansion medium, a maturation medium disclosed herein may lack one or more supplements that promotes a progenitor phenotype.

### Hepatocytes

In some aspects of the compositions and methods described herein, the hepatocytes are PHH. In some embodiments, culturing the PHH in an expansion method disclosed herein allows the cells to multiply, while retaining their hepatic phenotype. In some embodiments, populations of expanded PHH formed using the expansion method include hepatic stem or progenitor-like cells. In some aspects, the method further includes maturing a population of expanded PHH.

In some embodiments, the PHH are obtained from mature tissue. In some embodiments, the PHH are not derived from hepatocyte lines, e.g., which have been differentiated in vitro. In some embodiments, the PHH are, or are derived from, primary hepatocytes.

The PHH may be obtained by any suitable method. In some embodiments, cells are isolated by collagenase digestion, for example, as described in the examples and in Dorell et al., 2008 (Hepatology. 2008 48:1282-91). In some embodiments, collagenase digestion is performed on a tissue biopsy. In some embodiments, collagenase and accutase digestion are used to obtain the PHH.

PHH are present in the liver. In some embodiments, the method includes culturing a fragment of tissue which includes liver epithelium. In some embodiments, the PHH are isolated from a tissue fragment. For example, in the context of liver, the tissue fragment may include a liver biliary duct or biliary duct tissue. Liver PHH can be isolated from normal liver tissues using FACS-based sorting to exclude EpCAM⁺ progenitor cells. In some embodiments, PHH isolated from normal liver tissue may contain EpCAM⁺ progenitor cells.

In another embodiment, the cells of the invention may be isolated by immuno-affinity purification, which is a separation method well known in the art. By way of illustration only, the cells of the invention may be isolated by immuno-affinity purification directed towards c-kit. As will be apparent to one skilled in the art, this method relies upon the immobilization of antibodies on a purification column. The cell sample is then loaded onto the column, allowing the appropriate cells to be bound by the antibodies, and therefore bound to the column. Following a washing step, the cells are eluted from the column using a competitor which binds preferentially to the immobilized anti-c-kit antibody and permits the cells to be released from the column.

In some embodiments, the cells may be cultured after isolation for at least about 5, at least about 10, at least about 15, at least about 20 days, at least about 25 days, or at least about 30 days. In some embodiments, the cells may be cultured after isolation for at least about 5 days. In some embodiments, the cells may be cultured after isolation for at least about 10 days. In some embodiments, the cells may be cultured after isolation for at least about 15 days. In some embodiments, the cells may be cultured after isolation for at least about 20 days. In some embodiments, the cells may be cultured after isolation for at least about 25 days. In some embodiments, the cells may be cultured after isolation for at least about 25 days.

In certain aspects, the cells are expanded in culture longer to improve the homogeneity of the cell phenotype in the cell population or to stabilize the cell state of expanded cells.

In some embodiments, a population of cells may be used as the starting point, for example, a population of cells contained in a liver fragment as described above. Thus, the methods of the invention are not restricted to using single cells as the starting point.

In some embodiments, there is provided a method for obtaining a population of expanded PHH including culturing PHH in an expansion medium using the method as described herein.

In some embodiments, the method includes culturing the PHH or obtaining the population of expanded PHH from a single cell. Advantageously, this allows a homogenous population of cells to form. In some embodiments, the method includes culturing the PHH in an expansion medium of the invention for 3-120 days (e.g., 4-119 days, 5-118 days, 10-117 days, 15-116 days, 20-115 days, 30-100 days, 40-90 days, 50-80 days, 60-70 days, or 65 days), and then dissociating the cells to a single cell density, seeding one or more cells at a ratio of 1 cell per container (e.g., per well), and expanding the cells using an expansion medium of the invention.

For example, in some embodiments, the method includes culturing the PHH in an expansion medium of the invention for 3-120 days. In some embodiments, the method includes culturing the PHH in an expansion medium of the invention for 4-119 days. In some embodiments, the method includes culturing the PHH in an expansion method of the invention for 5-118 days. In some embodiments, the method includes culturing the PHH in an expansion method of the invention for 10-117 days. In some embodiments, the method includes culturing the PHH in an expansion method of the invention for 15-116 days. In some embodiments, the method includes culturing the PHH in an expansion method of the invention for 20-115 days. In some embodiments, the method includes culturing the PHH in an expansion method of the invention for 30-100 days. In some embodiments, the method includes culturing the PHH in an expansion method of the invention for 40-90 days. In some embodiments, the method includes culturing the PHH in an expansion method of the invention for 50-80 days. In some embodiments, the method includes culturing the PHH in an expansion method of the invention for 60-70 days. In some embodiments, the method includes culturing the PHH in an expansion method of the invention for 65 days.

In some embodiments, the culturing step includes expanding plated cells (step P0) and a first passage of expanded cells (step P1).

In some embodiments, the P0 step has a duration of between 7 to 16 days (e.g., 7, 8, 9, 10, 11, 12, 13, 14, 15, or 16 days). In some embodiments, the P0 step has a duration of 11 days. In some embodiments, the P0 step has a duration of 13 days.

In some embodiments, the P1 step has a duration of between 7 to 20 days (e.g., 7, 8, 9, 10, 11, 12, 13, 14, 15, 16, 17, 18, 19, or 20 days). In some embodiments, the P1 step has a duration of 11 days. In some embodiments, the P1 step has a duration of 13 days.

In some embodiments, the P0 step includes seeding the hepatocytes at a density of between 200 to 13,333 cells/cm², e.g., between 200 cells/cm² and 1,000 cells/cm² (e.g., 200 cells/cm², 300 cells/cm², 400 cells/cm², 500 cells/cm², 600 cells/cm², 700 cells/cm², 800 cells/cm², 900 cells/cm², or 1,000 cells/cm²), 1,000 cells/cm² to 10,000 cells/cm² (e.g., 1,000 cells/cm², 2,000 cells/cm², 3,000 cells/cm², 4,000 cells/cm², 5,000 cells/cm², 6,000 cells/cm², 7,000 cells/cm², 8,000 cells/cm², 9,000 cells/cm², or 10,000 cells/cm²), or 10,000 cells/cm² to 13,333 cells/cm² (e.g., 10,000 cells/cm², 11,000 cells/cm², 12,000 cells/cm², 13,000 cells/cm², or 13,333 cells/cm²). In some embodiments, the P0 step includes seeding the hepatocytes at a density of 667 cells/cm². In some embodiments, the P1 step includes seeding the hepatocytes at a density of between 333 to 13,333 cells/cm². In some embodiments, the P1 step includes seeding the hepatocytes at a density of 1,333 cells/cm².

In some embodiments, the PHH are cultured in the presence of about 5% carbon dioxide. In some embodiments, the PHH are cultured at a temperature of about 37° C. In some embodiments, PHH are cultured in hypoxic conditions.

Advantageously, use of the culture methods provided by the present invention results in cell populations being formed in which the hepatocytes remain non-tumorigenic when the cells are cultured long-term, as determined by molecular characterization of expanded cells. Thus, in some embodiments, the population of PHH of the invention remains non-tumorigenic after four or more (e.g., five, six, seven, or eight) months in culture in an expansion medium of the invention, as determined by molecular characterization of expanded cells. For example, in some embodiments, the population of PHH of the invention remains non-tumorigenic after five months in culture in an expansion medium of the invention, as determined by molecular characterization of expanded cells. In some embodiments, the population of PHH of the invention remains non-tumorigenic after six months in culture in an expansion medium of the invention, as determined by molecular characterization of expanded cells. In some embodiments, the population of PHH of the invention remains non-tumorigenic after seven months in culture in an expansion medium of the invention, as determined by molecular characterization of expanded cells. In some embodiments, the population of PHH of the invention remains non-tumorigenic after eight months in culture in an expansion medium of the invention, as determined by molecular characterization of expanded cells.

Following culturing, the method may further include obtaining and/or isolating one or more PHH. For example, following culture of the PHH, it may be useful to remove one or more PHH cultured in the expansion medium from the culture medium for use in subsequent applications. For example, it may be useful to isolate a single cell for culture using the expansion medium of the invention. Alternatively, it may be useful to obtain a population of cells for culture using the expansion medium of the invention.

The population of expanded PHH of the invention preferably includes at least 50% (e.g., at least 60%, 70%, 80%, or 90%) viable cells. For example, in some embodiments, the population of expanded PHH of the invention preferably includes at least 60% viable cells. In some embodiments, the population of expanded PHH of the invention preferably includes at least 70% viable cells. In some embodiments, the population of expanded PHH of the invention preferably includes at least 80% viable cells. In some embodiments, the population of expanded PHH of the invention preferably includes at least 90% viable cells. Viability of cells may be assessed using Hoechst staining or Propidium Iodide staining in FACS.

In some embodiments, there is provided one or more frozen populations of PHH of the invention. Also provided is a method for preparing populations of expanded PHH for freezing including dissociating expanded population of PHH cultures and mixing them with a freezing medium such as Recovery cell culture freezing medium (Gibco) or CryoStor (Biolife Solutions) and freezing following standard procedures. A method for thawing frozen PHH is also provided which includes thawing frozen PHH, embedding the thawed PHH in an ECM (e.g., collagen-I or Laminin-521) and culturing the PHH in an expansion medium of the invention.

In some examples, initially after thawing the culture medium may be supplemented with Y-27632, for example, about 10 UM Y-27632. In some embodiments, the culture medium is supplemented with Y-27632 for the first 1, 2, 3, 4, 5 or less days after thawing, preferably for the first 3 or 4 days. In some embodiments, Y-27632 is not present in the culture medium after the first 3, 4, 5, 6 or more days, preferably after the first 3 or 4 days. This thawing method can be used for expansion of PHH of the invention. In other examples, a ROCK inhibitor such as Y-27632 may be absent.

The cells produced by the methods described herein can be used immediately. Alternatively, the cells can be frozen at liquid nitrogen temperatures and stored for long periods of time, being thawed and capable of being reused. For example, the cells can be frozen in 10% dimethylsulfoxide (DMSO), 50% serum, 40% buffered medium, or some other such solution as is commonly used in the art to preserve cells at such freezing temperatures and thawed in a manner as commonly known in the art for thawing frozen cultured cells.

In some embodiments, the cells will expand at a rate of more than two (e.g., 3, 4, 5, 10, 20, 30) population doublings a week. For example, in some embodiments, the cells will expand at a rate of more than 3 population doublings a week. In some embodiments, the cells will expand at a rate of more than 4 population doublings a week. In some embodiments, the cells will expand at a rate of more than 5 population doublings a week. In some embodiments, the cells will expand at a rate of more than 10 population doublings a week. In some embodiments, the cells will expand at a rate of more than 20 population doublings a week. In some embodiments, the cells will expand at a rate of more than 30 population doublings a week.

In some embodiments, the method may include changing the medium for fresh medium during the course of culturing because the components of the medium are used up during culturing. It will be clear to the skilled person how often the medium needs to be changed for fresh medium. In some embodiments, the medium is changed every other day, but it is also envisaged that it may be changed every day or every two days or as required.

The expansion medium preferably induces or promotes the survival and/or proliferation of cells during at least 5 (e.g., at least 5, 10, 25, 50, or 100) days of culture. For example, in some embodiments, the expansion medium induces or promotes the survival and/or proliferation of cells during at least 10 days (e.g., at least 25 days). In some embodiments, the expansion medium induces or promotes the survival and/or proliferation of cells during at least 25 days. In some embodiments, the expansion medium induces or promotes the survival and/or proliferation of cells during at least 50 days. In some embodiments, the expansion medium induces or promotes the survival and/or proliferation of cells during at least 100 days.

Proliferation can be assessed using techniques known in the art, such as BrdU staining, Edu staining, Ki67 staining, and the use of growth curves assay can be done. For example, after thawing and plating 200 to 1000 cells per cm² frozen PHH in appropriate cell culture vessels for two-dimensional cell culture, at least 10-fold (e.g., at least 15-fold, 20-fold, or 30-fold) expansion of cells is achieved in passage 0 (P0) such that cell yield after expansion is 2×10³ to 10×10³ cells per cm². Upon reaching confluence, it is possible to subsequently passage expanded PHH and seed at 200 to 1000 cells per cm² or higher density until approximately at least 500-fold (e.g., at least 1000-fold) PHH expansion is achieved. In some embodiments, upon reaching confluence, it is possible to subsequently passage expanded PHH at a 1:3 ratio until approximately 100-fold PHH expansion is achieved. This is important for the industry since the availability of PHH for transplantation poses a significant problem. For a mouse transplant, for example, a minimum of 10⁵ cells are required. Possibly 1×10⁷ cells might be required for a human transplant, in order for a graft to be successful.

Put another way, expansion media used according to the invention are capable of expanding a population of PHH to form an expanded population of PHH maintaining the hepatic phenotype for at least 6 passages under appropriate conditions or achieving at least 500-fold expansion (e.g., at least 1000-fold expansion).

In some embodiments, following the culturing step, the expression profile of the PHH includes expression of Ki67 (i.e., protein) by at least 15% (e.g., at least 15%, 20%, 25%, 30%, 35%, 40%, 45%, 50%, 55%, 60%, 65%, 70%, 75%, or more) of the PHH. In some embodiments, following the culturing step, the expression profile of the PHH includes expression of Ki67 by at most 15% (e.g., at most 10%, 5%, 4%, 3%, 2%, or 1%) of the PHH.

In some embodiments, following the culturing step, the expression profile of the PHH includes expression of Ki67 (i.e., gene) by at least 15% (e.g., at least 15%, 20%, 25%, 30%, 35%, 40%, 45%, 50%, 55%, 60%, 65%, 70%, 75%, or more) of the PHH. In some embodiments, following the culturing step, the expression profile of the PHH includes expression of Ki67 by at most 15% (e.g., at most 10%, 5%, 4%, 3%, 2%, or 1%) of the PHH.

In some embodiments, following the culturing step, the hepatocyte yield expands by at least 2-fold (e.g., at least 3-fold, 4-fold, 5-fold, 10-fold, 20-fold, or 30-fold) within 14-28 days (e.g., within 15-27 days, within 16-26 days, within 17-25 days, within 18-24 days, within 19-23 days, within 20-21 days, or within 22 days) of culturing.

In some embodiments, following the culturing step, the hepatocyte yield expands by at least 500-fold within 30 days (e.g., within 30, 29, 28, 27, 26, 25, 24, 23, 22, 21, 20, 19, 18, 17, 16, 15, or 14 days) of culturing. In some embodiments, following the culturing step, the hepatocyte yield expands by at least 500-fold within 24 days of culturing.

In some embodiments, following the culturing step, the hepatocyte yield expands by between 500-fold to 2000-fold (e.g., 1000-fold to 2000-fold, e.g., 500-fold, 600-fold, 700-fold, 800-fold, 900-fold, 1,000-fold, 1,100-fold, 1,200-fold, 1,300-fold, 1,400-fold, 1,500-fold, 1,600-fold, 1,700-fold, 1,800-fold, 1,900-fold, or 2,000-fold) within 30 days (e.g., within 30, 29, 28, 27, 26, 25, 24, 23, 22, 21, 20, 19, 18, 17, 16, 15, or 14 days) of culturing. In some embodiments, following the culturing step, the hepatocyte yield expands by between 500-fold to 2000-fold (e.g., 1000-fold to 2000-fold, e.g., 500-fold, 600-fold, 700-fold, 800-fold, 900-fold, 1,000-fold, 1,100-fold, 1,200-fold, 1,300-fold, 1,400-fold, 1,500-fold, 1,600-fold, 1,700-fold, 1,800-fold, 1,900-fold, or 2,000-fold) within 24 days of culturing.

In some embodiments, following hepatocyte expansion, the expanded hepatocyte population may undergo maturation in the presence of a maturation medium including a basal medium for human cells to which is added one or more hepatocyte maturation supplements. In some embodiments, the maturation step immediately follows hepatocyte expansion. In some embodiments, the maturation step does not immediately follow hepatocyte expansion. In some embodiments, the maturation step has a duration of between 3 and 12 days (e.g., 3, 4, 5, 6, 7, 8, 9, 10, 11, or 12 days). In some embodiments, the maturation step is 7 days.

Maturation can be assessed using techniques known in the art, such as phase contrast microscopy to visualize cell morphology, detection of secreted urea and albumin, and RT-qPCR of hepatocyte maturation markers.

### Wnt Signaling Activator

The expansion medium of the invention includes one or more Wnt agonist. The Wnt signaling pathway is defined by a series of events that occur when the cell-surface Wnt receptor complex, including a Frizzled receptor, LRP and LGR is activated, usually be an extracellular signaling molecule, such as a member of the Wnt family. This results in the activation of Disheveled family proteins which inhibit a complex of proteins that includes axin, GSK-3, and the protein APC to degrade intracellular β-catenin. The resulting enriched nuclear β-catenin enhances transcription by TCF/LEF family transcription factors. A Wnt agonist is an agent that activates TCF/LEF-mediated transcription in a cell. Wnt agonists are therefore selected from Wnt agonists that bind and activate the Wnt receptor complex including any and all of the Wnt family proteins, such as an inhibitor of intracellular β-catenin degradation, a GSK inhibitor (such as CHIR9901) and activators of TCF/LEF.

In some embodiments, a Wnt agonist is a secreted glycoprotein selected from this list: Wnt-I/Int-1, Wnt-2/Irp (InM-related protein), Wnt-2b/13, Wnt-3/Int-4, Wnt-3a (R&D systems), Wnt-4, Wnt-5a, Wnt-5b, Wnt-6 (Kirikoshi H et al., 2001 Biochem Biophys Res Com 283 798-805), Wnt-7a (R&D systems), Wnt-7b, Wnt-8a/8d, Wnt-8b, Wnt-9a/14, Wnt-9b/14b/15, Wnt-10a, Wnt-10b/12, WnMI, and Wnt-16. An overview of human Wnt proteins is provided in “THE WNT FAMILY OF SECRETED PROTEINS,” R&D Systems Catalog, 2004.

The Wnt agonist in the expansion medium is preferably any agonist able to stimulate the Wnt pathway via the LGR5 cell surface receptor, e.g., in one embodiment, the Wnt agonist in the expansion medium is an LGR5 agonist. Known LGR5 agonists include R-spondin, fragments and derivatives thereof, and anti-LGR5 antibodies (e.g., See WO 2012/140274 and De Lau, W. et al. Nature, 2011 Jul. 4; 476 (7360): 293-7). A preferred LGR5 agonist is R-spondin. Any suitable R-spondin may be used, for example, any selected from the list including R-spondin 1, R-spondin 2, R-spondin 3, R-spondin 4, or derivatives thereof. For example, any of R-spondin 1 (NU206, Nuvelo, San Carlos, Calif.), R-spondin 2 ((R&D systems), R-spondin 3, and R-spondin-4 may be used.

The Wnt agonist is preferably added to the media in an amount effective to stimulate Wnt activity in a cell. As is known to a skilled person, Wnt activity can be determined by measuring the transcriptional activity of Wnt, for example by pTOPFLASH and pFOPFLASH Tcf luciferase reporter constructs (Korinek et al., 1997. Science 275:1784-1787).

In some embodiments, the secreted glycoprotein Wnt agonist is Wnt3a. The expansion media of the invention may include at least 10 ng/mL Wnt3a (e.g., at least 50 ng/mL, 100 ng/mL, 1 μg/mL, or 10 μg/mL). For example, in some embodiments, the media includes at least 50 ng/mL Wnt3a. In some embodiments, the media includes at least 100 ng/mL Wnt3a. In some embodiments, the media includes at least 1 μg/mL Wnt3a. In some embodiments, the media includes at least 10 μg/mL Wnt3a.

In some embodiments, the LGR5 agonist is selected from R-spondin1, R-spondin 2, R-spondin 3, or R-spondin 4. In some embodiments, the expansion medium includes at least 10 ng/mL (e.g., at least 50 ng/mL, 100 ng/mL, 1 μg/mL, or 5 μg/mL) R-spondin. For example, in some embodiments, the expansion medium includes at least 50 ng/mL R-spondin. In some embodiments, the expansion medium includes at least 100 ng/mL R-spondin. In some embodiments, the expansion medium includes at least 1 μg/mL R-spondin. In some embodiments, the expansion medium includes at least 5 μg/mL R-spondin.

In some embodiments, during culturing of PHH, the one or more Wnt agonist may be added to the culture medium when required, for example, daily or every other day. The Wnt agonist is preferably added to the culture medium every second day. In some embodiments, the Wnt agonist may be mixed with the culture medium prior to adding the medium to the cells.

### Receptor Tyrosine Kinase Ligand

The receptor tyrosine kinase ligands described herein include epidermal growth factor (EGF), fibroblast growth factor (FGF), hepatocyte growth factor (HGF), and transforming growth factor (TGF), and are preferably all present in the expansion medium. Many receptor tyrosine kinase ligands are mitogenic growth factors.

EGF is a protein that stimulates cell growth and differentiation by binding to its receptor, the epidermal growth factor receptor (EGFR).

FGF family members possess broad mitogenic and cell survival activities, and are involved in a variety of biological processes, including embryonic development, cell growth, morphogenesis, tissue repair, tumor growth and invasion. FGFs stimulate cells by interacting with cell surface fibroblast growth factor receptor (FGFR). Four closely related receptors (FGFR1-FGFR4) have been identified. Most FGFs bind more than one receptor (Ornitz J. Biol. Chem. 1998 Feb. 27; 273 (9): 5349-57). However, fibroblast growth factor 10 (FGF-10) and fibroblast growth factor 7 (FGF-7) are unique among FGFs in that they interact only with a specific isoform of FGFR2, designated FGFR2b, which is expressed exclusively by epithelial cells (Igarashi, J. Biol. Chem. 1998 273 (21): 13230-5).

Hepatocyte growth factor/scatter factor (HGF/SF) is a morphogenic factor that regulates cell growth, cell motility, and morphogenesis by activating a tyrosine kinase signaling cascade after binding to the proto-oncogenic c-Met receptor.

Transforming growth factor (TGF) is a polypeptide growth factor. One TGF, transforming growth factor-alpha (TGFα), binds to the EGFR and induces development of the epithelial phenotype.

In some embodiments, the one or more receptor tyrosine kinase ligands in the expansion medium are selected from the group consisting of: EGF, FGF, HGF, and TGF, wherein the FGF is preferably FGF-7 or FGF10.

In some embodiments, the expansion medium includes one or more receptor tyrosine kinase ligands. In some embodiments, only one receptor tyrosine kinase ligand is included in the expansion medium, which may be selected from FGF, HGF, EGF, and TGF. In some embodiments, the expansion medium includes two receptor tyrosine kinase ligands. In some embodiments, the two receptor tyrosine kinase ligands in the expansion medium are EGF and FGF. In some embodiments, the two receptor tyrosine kinase ligands in the expansion medium are EGF and HGF. In some embodiments, the two receptor tyrosine kinase ligands in the expansion medium are HGF and FGF. In some embodiments, the two receptor tyrosine kinase ligands in the expansion medium are TGF and FGF. In some embodiments, the two receptor tyrosine kinase ligands in the expansion medium are TGF and HGF. In some embodiments, the two receptor tyrosine kinase ligands in the expansion medium are EGF and TGF. In some embodiments, the expansion medium includes three receptor tyrosine kinase ligands. In some embodiments, the three receptor tyrosine kinase ligands in the expansion medium are EGF, FGF, and TGF. In some embodiments, the three receptor tyrosine kinase ligands in the expansion medium are EGF, FGF, and HGF. In some embodiments, the three receptor tyrosine kinase ligands in the expansion medium are TGF, HGF, and EGF. In some embodiments, the three receptor tyrosine kinase ligands in the expansion medium are TGF, HGF, and FGF. In some embodiments, the expansion medium includes four receptor tyrosine kinase ligands. In some embodiments, the four receptor tyrosine kinase ligands are EGF, FGF, HGF, and TGF.

In some embodiments, any suitable EGF may be used. In some embodiments, the EGF includes human EGF. In some embodiments, the EGF includes recombinant human EGF.

In some embodiments, EGF is added to the expansion medium at a concentration of between 5 and 500 ng/mL. For example, in some embodiments, the expansion medium includes 10 ng/mL EGF. In some embodiments, the expansion medium includes 50 ng/mL EGF. In some embodiments, the expansion medium includes 100 ng/mL EGF. In some embodiments, the expansion medium includes 1 μg/mL EGF. In some embodiments, the expansion medium includes 5 μg/mL EGF. In some embodiments, the expansion medium includes 10 μg/mL EGF. In some embodiments, EGF is substituted with an alternative compound that activates EGFR. For example, it is envisaged that insulin-like growth factor (IGF) may be substituted for EGF.

The FGF used in the expansion medium is preferably an FGF able to bind to fibroblast growth factor receptor 2 (FGFR2), and is preferably FGF-7 or FGF-10. In some embodiments, no more than one FGF is used. In some embodiments, the FGF is FGF-7. In some embodiments, the FGF is FGF-10. In other embodiments, two or more FGF are used, e.g., two, three, or more. In some embodiments, two FGF are used. In some embodiments, the two FGF are FGF-7 and FGF-10. In some embodiments, FGF is substituted with a compound that activates the FGFR2 pathway (a “FGF-pathway activator”). In some embodiments, the expansion medium includes 20-500 ng/mL FGF. For example, in some embodiments, the expansion medium includes 50 ng/mL FGF. In some embodiments, the expansion medium includes 100 ng/mL FGF. In some embodiments, the expansion medium includes 250 ng/mL FGF.

In some embodiments, the HGF is human HGF. In some embodiments, the HGF is recombinant human HGF. In some embodiments, the expansion medium includes 1-50 ng/mL HGF. For example, in some embodiments, the expansion medium includes 1 ng/mL HGF. In some embodiments, the expansion medium includes 5 ng/mL HGF. In some embodiments, the expansion medium includes 10 ng/mL HGF. In some embodiments, the expansion medium includes 25 ng/mL HGF. In some embodiments, the expansion medium includes 50 ng/mL HGF. In some embodiments, HGF is substituted with a compound that activates the HGF receptor, such as Dihexa.

In some embodiments, the FGF is human FGF. In some embodiments, the FGF is recombinant human FGF-7 and recombinant human FGF-10. In some embodiments, the expansion medium includes at least 5 ng/mL FGF. For example, in some embodiments, the expansion medium includes 10 ng/mL FGF. In some embodiments, the expansion medium includes 50 ng/mL FGF. In some embodiments, the expansion medium includes 100 ng/mL FGF. In some embodiments, the expansion medium includes 250 ng/mL FGF.

In some embodiments, the TGF is human TGFα. In some embodiments, the TGF is recombinant human TGFα. In some embodiments, the expansion medium includes at least 2 ng/mL TGF. For example, in some embodiments, the expansion medium includes 2.5 ng/mL TGF. In some embodiments, the expansion medium includes 5 ng/mL TGF. In some embodiments, the expansion medium includes 10 ng/mL TGF. In some embodiments, the expansion medium includes 50 ng/mL TGF. In some embodiments, the expansion medium includes 100 ng/mL TGF. In some embodiments, the expansion medium includes 250 ng/mL TGF.

In some embodiments, during culturing of PHH, the one or more receptor tyrosine kinase ligand (e.g., EGF, FGF-7, FGF-10, HGF, and TGFα) is added to the culture medium when required, for example, daily or every other day. They may be added singularly or in combination. It is preferable that they are added every second day. In some embodiments, the one or more receptor tyrosine kinase ligand may be added directly to the cell culture medium prior to addition to the cells.

### Epithelial Phenotype Stabilizing Agent

The expansion medium described herein may include one or more epithelial phenotype stabilizing agent. In some embodiments, the one or more epithelial phenotype stabilizing agent is a transforming growth factor-beta (TGFβ) inhibitor. The presence of a TGFβ inhibitor in the expansion media is advantageous because it prevents the PHH from differentiating. In other words, the TGFβ inhibitor reduces or inhibits the activity of the TGFβ signaling pathway, thereby preventing development of the mesenchymal phenotype. TGFβ signaling is involved in many cellular functions, including cell growth, cell fate and apoptosis. Signaling typically begins with binding of a TGFβ superfamily ligand to a type II receptor which recruits and phosphorylates a type I receptor. The type I receptor then phosphorylates SMADs, which act as transcription factors in the nucleus and regulate target gene expression.

The TGFβ superfamily ligands include bone morphogenic proteins (BMPs), growth and differentiation factors (GDFs), anti-müllerian hormone (AMH), activin, nodal and TGFβs. In general, Smad2 and Smad3 are phosphorylated by the ALK4, 5 and 7 receptors in the TGFβ/activin pathway. By contrast, Smad1, Smad5 and Smad8 are phosphorylated as part of the bone morphogenetic protein (BMP) pathway. Although there is some cross-over between pathways, in the context of this invention, a “TGFβ inhibitor” or an “inhibitor of TGFβ Signaling” is preferably an inhibitor of the TGFβ pathway which acts via Smad2 and Smad3. Therefore, in some embodiments the TGFβ inhibitor is not a BMP inhibitor, e.g., the TGFβ inhibitor is not Noggin. In some embodiments, a BMP inhibitor is added to the culture medium in addition to the TGFβ inhibitor (see below).

Thus, the TGFβ inhibitor may be any agent that reduces the activity of the TGFβ signaling pathway. There are many ways of disrupting the TGFβ signaling pathway that are known in the art and that can be used in conjunction with this invention. For example, the TGFβ signaling may be disrupted by: inhibition of TGFβ expression by a small-interfering RNA strategy; inhibition of furin (a TGFβ activating protease); inhibition of the pathway by physiological inhibitors; neutralization of TGFβ with a monoclonal antibody; inhibition with small-molecule inhibitors of TGFβ receptor kinase 1 (also known as ALK5), or other TGFβ-related receptor kinases; inhibition of Smad 2 and Smad 3 signaling e.g. by overexpression of their physiological inhibitor, Smad 7, or by using thioredoxin as an Smad anchor disabling Smad from activation (Fuchs, O. Inhibition of TGF-Signaling for the Treatment of Tumor Metastasis and Fibrotic Diseases. Current Signal Transduction Therapy, Volume 6, Number 1, January 2011, pp. 29-43 (15)).

Various methods for determining if a substance is a TGFβ inhibitor are known and might be used in conjunction with the invention. For example, a cellular assay may be used in which cells are stably transfected with a reporter construct including the human PAI-1 promoter or Smad binding sites, driving a luciferase reporter gene. Inhibition of luciferase activity relative to control groups can be used as a measure of compound activity (De Gouville et al., Br J Pharmacol. 2005 May; 145 (2): 166-177). A TGFβ inhibitor according to the present invention may be a protein, peptide, small-molecules, small-interfering RNA, antisense oligonucleotide, aptamer, or antibody. The inhibitor may be naturally occurring or synthetic. In one embodiment, the TGFβ inhibitor is an inhibitor of ALK5. For example, the TGFβ inhibitor may bind to and directly inhibit ALK5. Examples of preferred small-molecule TGFβ inhibitors that can be used in the context of this invention include A83-01. In some embodiments, the TGFβ inhibitor is a small molecule inhibitor. In some embodiments, the TGFβ inhibitor is A83-01.

In some embodiments, no more than one TGFβ inhibitor is present in the expansion medium. In other embodiments, more than one (e.g., two, three, four, or more) TGFβ inhibitor is present in the expansion medium. The skilled person will appreciate that a number of other small-molecule inhibitors exist that are primarily designed to target other kinases, but at high concentrations that may also inhibit TGFβ signaling pathway also can be used in the context of this invention.

In some embodiments, the TGFβ inhibitor is an ALK5 inhibitor. In some embodiments, the ALK5 inhibitor is A83-01. In some embodiments, the expansion medium includes at least 10 nM (e.g., at least 50 nM, 100 nM, 1 μM, 5 μM, or 10 μM). For example, in some embodiments, the expansion medium includes at least 50 nM A83-01. In some embodiments, the expansion medium includes at least 100 nM A83-01. In some embodiments, the expansion medium includes at least 1 μM A83-01. In some embodiments, the expansion medium includes at least 5 μM A83-01. In some embodiments, the expansion medium includes at least 10 μM A83-01.

In some embodiments, the epithelial phenotype stabilizing agent may further include a corticosteroid. In some embodiments, the corticosteroid includes hydrocortisone. In some embodiments, the expansion medium includes at least 10 ng/mL (e.g., at least 25 ng/mL, 50 ng/mL, 100 ng/mL, 500 ng/mL, 1 μg/mL, 5 μg/mL, or 10 g/mL) hydrocortisone. For example, in some embodiments, the expansion medium includes at least 25 ng/mL hydrocortisone. In some embodiments, the expansion medium includes at least 50 ng/mL hydrocortisone. In some embodiments, the expansion medium includes at least 100 ng/mL hydrocortisone. In some embodiments, the expansion medium includes at least 250 ng/mL hydrocortisone. In some embodiments, the expansion medium includes at least 500 ng/mL hydrocortisone. In some embodiments, the expansion medium includes at least 1 g/mL hydrocortisone. In some embodiments, the expansion medium includes at least 5 μg/mL hydrocortisone. In some embodiments, the expansion medium includes at least 10 μg/mL.

In some embodiments, during culturing of PHH, the one or more epithelial phenotype stabilizing agent is added to the culture medium when required, for example, daily or every other day. It is preferable that it is added every second day. In some embodiments, the one or more epithelial phenotype stabilizing agent may be added directly to the cell culture medium prior to addition to the PHH.

### Promotion of Cell Survival and Proliferation

An expansion medium described herein optionally includes one or more agents that promote cell survival and/or proliferation selected from the group consisting of N-acetylcysteine, B27, N2, nicotinamide, and a Rho kinase inhibitor. N-acetylcysteine, B27, N2, nicotinamide, and Rho kinase inhibitors are believed to control cell survival, cell proliferation, and assist with DNA stability.

In some embodiments, the expansion medium is supplemented with at least 0.25 mM (e.g., at least 0.5 mM, 1 mM, or 5 mM) N-acetylcysteine. For example, in some embodiments, the expansion medium is supplemented with at least 0.5 mM N-acetylcysteine. In some embodiments, the expansion medium is supplemented with at least 1 mM N-acetylcysteine. In some embodiments, the expansion medium is supplemented with at least 5 mM N-acetylcysteine.

In some embodiments, the B27 optionally does not contain vitamin A. The B27 supplement may be used to formulate a culture medium that includes biotin, cholesterol, linoleic acid, linolenic acid, progesterone, putrescine, retinyl acetate, sodium selenite, tri-iodothyronine (T3), DL-alpha tocopherol (vitamin E), albumin, insulin, and transferrin. The B27 supplement minus vitamin A was shown to work particularly well in the expansion medium for the liver. The B27 supplement comes as a liquid 50× concentrate, containing amongst other ingredients biotin, cholesterol, linoleic acid, linolenic acid, progesterone, putrescine, retinol, retinyl acetate, sodium selenite, tri-iodothyronine (T3), DL-alpha tocopherol (vitamin E), albumin, insulin, and transferrin. Of these ingredients, at least linolenic acid, retinol, retinyl acetate, and tri-iodothyronine (T3) are nuclear hormone receptor agonists. B27 supplement may be added to a culture medium as a concentrate or diluted before addition to a culture medium as a concentrate or diluted before addition to a culture medium. It may be used at a 1× final concentration or at other final concentrations. Use of B27 supplement is a convenient way to incorporate biotin, cholesterol, linoleic acid, linolenic acid, progesterone, putrescine, retinol, retinyl acetate, sodium selenite, tri-iodothyronine (T3), DL-alpha tocopherol (vitamin E), albumin, insulin, and transferrin into a culture medium of the invention. It is also envisaged that some or all of these components may be added separately to the expansion medium instead of using the B27 supplement. Thus, the expansion medium may include some or all of these components.

In some embodiments, the amount of B27 in the expansion medium can be between about 0.1× and about 100× (e.g., between about 0.1× and about 90×, between about 0.5× and about 80×, between about 1× and about 70×, between about 5× and 60×, and between about 10× and 50×).

For example, in some embodiments, the concentration of B27 in the expansion medium is about 0.1×. In some embodiments, the concentration of B27 in the expansion medium is about 0.5×. In some embodiments, the concentration of B27 in the expansion medium is about 1×. In some embodiments, the concentration of B27 in the expansion medium is about 5×. In some embodiments, the concentration of B27 in the expansion medium is about 10×. In some embodiments, the concentration of B27 in the expansion medium is about 50×. In some embodiments, the concentration of B27 in the expansion medium is about 70×. In some embodiments, the concentration of B27 in the expansion medium is about 80×. In some embodiments, the concentration of B27 in the expansion medium is about 90×. In some embodiments, the concentration of B27 in the expansion medium is about 100×.

The N2 supplement comes as a 100× liquid concentrate, containing human transferrin, bovine insulin, progesterone, putrescine, and sodium selenite. N2 supplement may be added to a culture medium as a concentrate or diluted before addition to a culture medium. It may be used at a 1× final concentration or at other final concentrations. Use of N2 supplement is a convenient way to incorporate transferrin, insulin, progesterone, putrescine, and sodium selenite into a culture medium of the invention. It is of course also envisaged that some or all of these components may be added separately to the expansion medium instead of using the N2 supplement. Thus, in some embodiments, the expansion medium may include some or all of these components.

In some embodiments, the amount of N2 in the expansion medium can be between about 0.1× and about 100× (e.g., between about 0.1× and about 90×, between about 0.5× and about 80×, between about 1× and about 70×, between about 5× and 60×, and between about 10× and 50×).

For example, in some embodiments, the concentration of N2 in the expansion medium is about 0.1×. In some embodiments, the concentration of N2 in the expansion medium is about 0.5×. In some embodiments, the concentration of N2 in the expansion medium is about 1×. In some embodiments, the concentration of N2 in the expansion medium is about 5×. In some embodiments, the concentration of N2 in the expansion medium is about 10×. In some embodiments, the concentration of N2 in the expansion medium is about 50×. In some embodiments, the concentration of N2 in the expansion medium is about 70×. In some embodiments, the concentration of N2 in the expansion medium is about 80×. In some embodiments, the concentration of N2 in the expansion medium is about 90×. In some embodiments, the concentration of N2 in the expansion medium is about 100×.

In some embodiments in which the medium includes B27, it does not include N2. The embodiments of the present invention can therefore be adapted to exclude N2 when B27 is present, if desired.

In some embodiments, N2 is not present in the expansion medium.

In some embodiments in which the medium includes N2, it also does not include B27. The embodiments of the present invention can therefore be adapted to exclude B27 when N27 is present, if desired.

In some embodiments, B27 is not present in the expansion medium.

In some embodiments, the expansion medium is supplemented with B27 and/or N2.

In some embodiments, the expansion medium includes at least 1 mM (e.g., at least 5 mM, 10 mM, or 50 mM) nicotinamide. For example, in some embodiments, the expansion medium includes at least 5 mM nicotinamide. In some embodiments, the expansion medium includes at least 10 mM nicotinamide. In some embodiments, the expansion medium includes at least 50 mM nicotinamide.

In some embodiments, the Rho kinase inhibitor is Y-27632. In some embodiments, the expansion medium includes at least 1 μM (e.g., at least 5 μM, 10 μM, or 50 μM) Y-27632. For example, in some embodiments, the expansion medium includes at least 5 μM Y-27632. In some embodiments, the expansion medium includes at least 10 μM Y-27632. In some embodiments, the expansion medium includes at least 50 μM Y-27632. In some embodiments, a Rho kinase inhibitor is not included in the expansion medium.

In some embodiments, during culturing of PHH, the one or more agent that promotes cell survival and/or proliferation selected from the group consisting of N-acetylcysteine, B27, N2, nicotinamide, and a Rho kinase inhibitor is added to the culture medium when required, for example, daily or every other day. It is preferable that it is added every second day. In some embodiments, the one or more agent that promotes cell survival and/or proliferation may be added directly to the cell culture medium prior to addition to the PHH.

In some embodiments, a maturation medium disclosed herein does not include one or more agent that promotes cell survival and/or proliferation, e.g., any of the agents that promotes cell survival and/or proliferation disclosed herein.

### Promotion of Maturation

The hepatocytes described herein may be matured according to a method as described herein. In some embodiments, the hepatocytes were previously expanded by a method as described herein. In some embodiments, the hepatocytes are expanded by a method as described herein and subsequently matured. A maturation medium described herein may include a basal medium for human cells. In some embodiments, the maturation basal medium is LONZA™ HCM™, William's E, or HepatoZYME-SFM. LONZA™ HCM™ is a hepatocyte culture medium that includes or is obtained by combining HBM™ basal medium with transferrin, ascorbic acid, human epidermal growth factor (HEGF), insulin, hydrocortisone, fatty acid free bovine serum albumin, and gentamicin sulfate-amphotericin (GA-1000). William's E medium is a reduced-serum supplemented medium for growing primary hepatocytes. HepatoZYME-SFM is a serum-free medium for the long-term maintenance of hepatocytes.

A maturation medium described herein optionally includes one or more agents that promote hepatocyte maturation selected from the group consisting of an antibiotic, HEPES, GLUTAMAX™, ITS, a Notch inhibitor, an EGFR inhibitor, Oncostatin M, an anti-oxidant, a glucocorticoid, a pregnane X receptor (PXR) activator, a bile acid, a cAMP or cAMP analog, cholesterol, a thyroid hormone, a serum replacement component, or any combination of the foregoing.

In some embodiments of the foregoing aspect, the Notch inhibitor is Compound E, Gamma Secretase Inhibitor XX, or a combination thereof. In some embodiments of the foregoing aspect, the antibiotic is penicillin, streptomycin, or a combination thereof. In some embodiments of the foregoing aspect, the EGFR inhibitor is erlotinib HCl. In some embodiments of the foregoing aspect, the anti-oxidant is vitamin C. In some embodiments of the foregoing aspect, the glucocorticoid is dexamethasone, hydrocortisone, or a combination thereof. In some embodiments of the foregoing aspect, the PXR activator is vitamin K2. In some embodiments of the foregoing aspect, the bile acid is lithocholic acid, urso deoxycholic acid, or a combination thereof. In some embodiments of the foregoing aspect, the CAMP analog is 8 bromo CAMP, forskolin, or a combination thereof. In some embodiments of the foregoing aspect, the thyroid hormone is T3. In some embodiments of the foregoing aspect, the serum replacement component is ITS, KOSR, Trace Elements A, Trace Elements B, or a combination thereof.

The maturation medium described herein may undergo supplement removal, e.g., relative to an expansion medium disclosed herein, to prevent cell proliferation or the progenitor phenotype. In some embodiments of any of the foregoing aspects, the maturation medium lacks one or more of R-spondin 1/Wnt3a, Epidermal Growth Factor (EGF), Transforming Growth Factor α (TGFα), N-acetyl cysteine, Nicotinamide, B27 supplement, N2 supplement, Fibroblast Growth Factor 7 (FGF7), and Fibroblast Growth Factor 10 (FGF10).

### Serum Replacement Components

The basal medium or maturation medium described herein may additionally include a serum replacement component.

Any suitable serum replacement component may be used. In some embodiments, the serum replacement component is KNOCKOUT™ Serum Replacement (KOSR), human platelet lysate, human serum, ITS, Trace Elements A, or Trace Elements B. KOSR is a serum-free eukaryotic cell culture medium supplement that includes or is obtained by combining albumin or an albumin supplement and one or more ingredients selected from the group consisting of glycine, L-histidine, L-isoleucine, L-methionine, L-phenylalanine, L-proline, L-hydroxyproline, L-serine, L-threonine, L-tryptophan, L-tyrosine, L-valine, thiamine, reduced glutathione, L-ascorbic acid-2-phosphate, iron saturated transferrin, insulin, and compounds containing the trace element moieties Ag⁺, Al³⁺, Ba²⁺, Cd²⁺, Co²⁺, Cr³⁺, Ge⁴⁺, Se⁴⁺, Br⁻, I⁻, Mn²⁺, F⁻, Si⁴⁺, V⁵⁺, Mo⁶⁺, Ni²⁺, Rb⁺, Sn²⁺ and Zr⁴⁺ (see, e.g., US Pub. No. US20020076747, the disclosure of which is hereby incorporated by reference in its entirety).

In some embodiments, the culturing step includes expanding plated cells (step P0) and a first passage of expanded cells (step P1). In some embodiments, the P0 step has a duration of between 7 to 16 days (e.g., 7, 8, 9, 10, 11, 12, 13, 14, 15, or 16 days). In some embodiments, the P0 step has a duration of 11 days. In some embodiments, the P0 step has a duration of 13 days.

In some embodiments, the P1 step has a duration of between 7 to 20 days (e.g., 7, 8, 9, 10, 11, 12, 13, 14, 15, 16, 17, 18, 19, or 20 days). In some embodiments, the P1 step has a duration of 11 days. In some embodiments, the P1 step has a duration of 13 days.

In some embodiments, the P0 step includes seeding the hepatocytes at a density of between 200 to 13,333 cells/cm², e.g., between 200 cells/cm² and 1,000 cells/cm² (e.g., 200 cells/cm², 300 cells/cm², 400 cells/cm², 500 cells/cm², 600 cells/cm², 700 cells/cm², 800 cells/cm², 900 cells/cm², or 1,000 cells/cm²), 1,000 cells/cm² to 10,000 cells/cm² (e.g., 1,000 cells/cm², 2,000 cells/cm², 3,000 cells/cm², 4,000 cells/cm², 5,000 cells/cm², 6,000 cells/cm², 7,000 cells/cm², 8,000 cells/cm², 9,000 cells/cm², or 10,000 cells/cm²), or 10,000 cells/cm² to 13,333 cells/cm² (e.g., 10,000 cells/cm², 11,000 cells/cm², 12,000 cells/cm², 13,000 cells/cm², or 13,333 cells/cm²). In some embodiments, the P0 step includes seeding the hepatocytes at a density of 667 cells/cm². In some embodiments, the P1 step includes seeding the hepatocytes at a density of between 333 to 13,333 cells/cm². In some embodiments, the P1 step includes seeding the hepatocytes at a density of 1,333 cells/cm².

In some embodiments, the expansion medium includes a serum replacement component, and the concentration of the serum replacement component is varied over the duration of the culturing step.

In some embodiments, the concentration of the serum replacement component is 1% (v/v) on Day 0 of the P0 step.

In some embodiments, the concentration of the serum replacement is raised to 5% (v/v) (i) when the cell density reaches between 15% to 30% (e.g., 15%, 20%, 25%, or 30%) confluency or (ii) between Day 3 to Day 7 (e.g., Day 3, Day 4, Day 5, Day 6, or Day 7) of the P0 step. In some embodiments, the concentration of the serum replacement is raised to 5% (v/v) on Day 5 of the P0 step. In some embodiments, the concentration of the serum replacement component is 5% (v/v) on Day 0 of the P0 step. In some embodiments, the concentration of the serum replacement component is 5% (v/v) on Day 0 of the P0 and remains 5% (v/v) until the concentration of the serum replacement component is increased. In some embodiments, the concentration of the serum replacement is raised to 10% (v/v) (i) when the cell density reaches between 40% to 60% (e.g., 40%, 45%, 50%, 55%, or 60%) confluency or (ii) between Day 7 to Day 13 (e.g., Day 7, Day 8, Day 9, Day 10, Day 11, Day 12, or Day 13) of the P0 step. In some embodiments, the concentration of the serum replacement is raised to 10% (v/v) on Day 9 of the P0 step.

In some embodiments, the concentration of the serum replacement component is 1% (v/v) on Day 0 of the P1 step. In some embodiments, the concentration of the serum replacement is raised to 5% (v/v) (i) when the cell density reaches between 15% to 30% % (e.g., 15%, 20%, 25%, or 30%) confluency or (ii) between Day 3 to Day 7 (e.g., Day 3, Day 4, Day 5, Day 6, or Day 7) of the P1 step. In some embodiments, the concentration of the serum replacement is raised to 5% (v/v) on Day 5 of the P1 step.

In some embodiments, the concentration of the serum replacement component is 5% (v/v) on Day 0 of the P1 step. In some embodiments, the concentration of the serum replacement component is 5% (v/v) on Day 0 of the P1 step and remains 5% (v/v) until the concentration of the serum replacement component is increased.

In some embodiments, the concentration of the serum replacement is raised to 10% (v/v) (i) when the cell density reaches between 40% to 60% (e.g., 40%, 45%, 50%, 55%, or 60%) confluency or (ii) between Day 5 to Day 13 (e.g., Day 5, Day 6, Day 7, Day 8, Day 9, Day 10, Day 11, Day 12, or Day 13) of the P1 step. In some embodiments, the concentration of the serum replacement is raised to 10% (v/v) on Day 9 of the P1 step. In some embodiments, the concentration of the serum replacement is raised to 10% (v/v) on Day 7 of the P1 step.

### Additional Components

In some examples, a basal medium described herein may additionally include a buffering agent. In some embodiments, the buffering agent is-2-hydroxyethylpiperazine-N′-2-ethanesulfonic acid (HEPES). In some embodiments, the expansion medium includes at least 1 mM (e.g., 5 mM, 10 mM, or 50 mM) HEPES. For example, in some embodiments, the expansion medium includes 5 mM HEPES. In some embodiments, the expansion medium includes 10 mM HEPES. In some embodiments, the expansion medium includes 50 mM HEPES.

In some embodiments, the basal medium includes L-glutamine or a derivative thereof.

In some embodiments, the L-glutamine is an L-glutamine supplement, e.g., L-alanyl-L-glutamine dipeptide, e.g., 0.200 mM L-alanyl-L-glutamine dipeptide in 0.85% NaCl (e.g., GLUTAMAX™). In some embodiments, the expansion medium includes at least 0.1% (e.g., at least 0.5%, 1%, or 5%) GLUTAMAX™. For example, in some embodiments, the expansion medium includes at least 0.5% GLUTAMAX™. In some embodiments, the expansion medium includes at least 1% GLUTAMAX™. In some embodiments, the expansion medium includes at least 5% GLUTAMAX™. In some embodiments, the expansion medium includes from 0.1% to 10%, e.g., 0.1% to 1% (e.g., 0.1%, 0.2%, 0.3%, 0.4%, 0.5%, 0.6%, 0.7%, 0.8%, 0.9%, or 1%) or 1% to 10% (e.g., 1%, 2%, 3%, 4%, 5%, 6%, 7%, 8%, 9%, or 10%) GLUTAMAX™.

In some embodiments, the basal medium additionally includes an antibiotic.

In some embodiments, the antibiotic includes, but is not limited to, a solution of penicillin and streptomycin. In some embodiments, the expansion medium or the maturation medium includes at least 0.1% (e.g., at least 0.5%, 1%, or 5%) of a solution of penicillin and streptomycin. For example, in some embodiments, the expansion medium or the maturation medium includes at least 0.5% of a solution of penicillin and streptomycin. In some embodiments, the expansion medium or the maturation medium includes at least 1% of a solution of penicillin and streptomycin. In some embodiments, the expansion medium or the maturation medium includes at least 5% of a solution of penicillin and streptomycin. In some embodiments, the expansion medium or the maturation medium includes from 0.1% to 10%, e.g., 0.1% to 1% (e.g., 0.1%, 0.2%, 0.3%, 0.4%, 0.5%, 0.6%, 0.7%, 0.8%, 0.9%, or 1%) or 1% to 10% (e.g., 1%, 2%, 3%, 4%, 5%, 6%, 7%, 8%, 9%, or 10%) a solution of penicillin and streptomycin.

In some embodiments, the expansion medium further includes an amino acid supplement. In some embodiments, the amino acid supplement is a non-essential amino acid (NEAA) supplement. In some embodiments, the NEAA supplement includes glycine, L-alanine, L-asparagine, L-aspartic acid, L-glutamic acid, L-proline, and L-serine. In some embodiments, the expansion medium includes a non-essential amino acid supplement containing, e.g., from 1 μM to 100 mM, e.g., from 1 μM to 10 μM (e.g., 1 μM, 2 μM, 3 μM, 4 μM, 5 μM, 6 μM, 7 μM, 8 μM, 9 μM, or 10 μM), 10 μM to 100 μM (e.g., 10 μM, 20 μM, 30 μM, 40 μM, 50 μM, 60 μM, 70 μM, 80 μM, 90 μM, or 100 μM), 100 μM to 1 mm (e.g., 100 μM, 200 μM, 300 μM, 400 μM, 500 μM, 600 μM, 700 M, 800 μM, 900 M, or 1 mm), 1 mM to 10 mM (e.g., 1 mM, 2 mM, 3 mM, 4 mM, 5 mM, 6 mM, 7 mM, 8 mM, 9 mM, or 10 mM), or 10 mM to 100 mM (e.g., 10 mM, 20 mM, 30 mM, 40 mM, 50 mM, 60 mM, 70 mM, 80 mM, 90 mM, or 100 mM) of each of glycine, L-alanine, L-asparagine, L-aspartic acid, L-glutamic acid, L-proline, and L-serine. In some embodiments, the expansion medium includes 100 μM of each of glycine, L-alanine, L-asparagine, L-aspartic acid, L-glutamic acid, L-proline, and L-serine.

In some embodiments, the expansion medium does not include a NEAA supplement.

In some embodiments, a serum is added to the expansion medium. In some embodiments, the serum includes, but is not limited to, fetal bovine serum. In some embodiments, the expansion medium includes at least 1% (e.g., at least 5%, 10%, or 15%) fetal bovine serum. For example, in some embodiments, the expansion medium includes at least 5% fetal bovine serum. In some embodiments, the expansion medium includes at least 10% fetal bovine serum. In some embodiments, the expansion medium includes at least 15% fetal bovine serum. In some embodiments, a serum is absent from the expansion medium.

In some embodiments, during culturing of PHH, the aforementioned agents are optionally added to the culture medium when required, for example, daily or every other day. It is preferable that they are added every second day. In some embodiments, the aforementioned agents may be added directly to the cell culture medium prior to addition to the PHH.

In some embodiments, a Notch inhibitor is absent from the expansion medium. In some embodiments, a Notch agonist is absent from the expansion medium. In some embodiments, neither a Notch inhibitor nor a Notch agonist are present in the expansion medium.

### Hypoxic Conditions

The culture methods described herein may include culturing PHH under hypoxic conditions or in the presence of a hypoxic mimetic. Hypoxic conditions, as described herein, include any condition where oxygen is present in concentrations below normal oxygen concentrations (normoxic conditions). Hypoxic mimetics mimic hypoxia by inducing the accumulation of hypoxia-inducible factor one alpha (HiF1α), which is a protein subunit of a transcription factor that responds to decreases in available oxygen. In some embodiments, culturing includes culturing the cells under hypoxic conditions. Hypoxic conditions may include, e.g., an oxygen level of less than 20%. In some embodiments, the culturing under hypoxic conditions includes culturing the cells at an oxygen level of between 1% to 19% (e.g., between 1% and 10%, e.g., 1%, 2%, 3%, 4%, 5%, 6%, 7%, 8%, 9%, 10%, 11%, 12%, 13%, 14%, 15%, 16%, 17%, 18%, 19%, or 20%). In some embodiments, the culturing under hypoxic conditions includes culturing the cells at an oxygen level of between 1% to 19%. In some embodiments, the culturing includes culturing the cells at an oxygen level of between 1% to 10%. In some embodiments, the culturing includes culturing the cells at an oxygen level of 5%. In some embodiments, the culturing includes culturing the cells under normoxic conditions.

In some embodiments, culturing includes culturing the cells in the presence of a hypoxic mimetic (e.g., a HIF-1α stabilizer or a PHD inhibitor). Exemplary hypoxia mimetics include, but are not limited to, an iron chelator (e.g., deferoxamine mesylate (DFO), compound A, deferasirox, and 2,2′-dipyridyl (DP)), an ion competitor (e.g., cobalt chloride (CoCl₂) or a divalent metal ion such as Ni²⁺, Mn²⁺, Co²⁺, or Zn²⁺), and a 2 oxoglutarate (20G) analog (e.g., dihydroxybenzoic acid (DHB), N-oxalylglycine, dimethyloxalylglycine (DMOG)), a PHD inhibitor (e.g., FG-4497, GSK360A, TM6008, or folic acid), or a HIF-1α stabilizer (e.g., miR-335, isoflurane, N-acetylcysteine, MG-132, BSc2118, and tilorone). Hypoxia mimetics are also described, e.g., in Davis et al. Front. Cell Dev. Biol. 6:175, 2018. In some embodiments, the culturing in the presence of a hypoxic mimetic includes culturing the cells in the presence of cobalt chloride. In some embodiments, the culturing in the presence of a hypoxic mimetic includes culturing the cells in the presence of desferrioxamine (e.g., DFO). In some embodiments, the culturing in the presence of a hypoxic mimetic includes culturing the cells in the presence of DMOG. In some embodiments, the culturing includes culturing the cells in the absence of a hypoxic mimetic.

### Culture Surface

In some examples of the methods described herein, the PHH are cultured on a two-dimensional surface. The cell culture surface can be made of any material suitable for culturing mammalian cells. For example, the surface can be a material that can be easily sterilized such as plastic or other artificial polymer material, so long as the material is biocompatible. In some embodiments, the cell culture surface may contain plastic or glass. In some embodiments, the surface includes any material that allows cells to adhere. In some embodiments, the cells are grown in one plane.

Any number of materials can be used to form the surface, including but not limited to, polyamides; polyesters; polystyrene; polypropylene; polyacrylates; polyvinyl compounds (e.g., polyvinylchloride); polycarbonate; polytetrafluoroethylene (PTFE); nitrocellulose; cotton; polyglyolic acid (PGA); cellulose; dextran; gelatin; glass; fluoropolymers; fluorinated ethylene propylene; polyvinylidene; polydimethylsiloxane; and silicon substrates (such as fused silica, polysilicon, or single silicon crystals), and the like. Also, metals (e.g., gold, silver, titanium films) can be used.

In some embodiments, the surface may be modified to promote cellular adhesion (e.g., coated with an adherence material). For example, a glass surface may be treated with a protein (i.e., a peptide of at least two amino acids) such as collagen or fibronectin to assist cells of the tissue in adhering to the substrate. In some embodiments, a single protein is adhered to the surface. In some embodiments, two or more proteins are adhered to the surface. Proteins suitable for use in modifying the substrate to facilitate adhesion include proteins to which specific cell types adhere under cell culture conditions. In some embodiments, the surface is coated with an ECM to facilitate cell adhesion.

In some embodiments, the PHH are cultured on a two-dimensional surface, wherein the two-dimensional surface includes a surface area of between 9.5 cm² to 10,000 cm². In some embodiments the PHH are cultured on a two-dimensional surface, wherein the two-dimensional surface includes a surface area of between 500 cm² to 10,000 cm². For example, in some embodiments, the surface area is between 9.5 cm² to 500 cm² (e.g., 9.5 cm², 100 cm², 200 cm², 300 cm², 400 cm², or 500 cm²), 500 cm² and 1,000 cm² (e.g., 500 cm², 600 cm², 700 cm², 800 cm², 900 cm², or 1,000 cm²), or 1,000 cm² to 10,000 cm² (e.g., 1,000 cm², 2,000 cm², 3,000 cm², 4,000 cm², 5,000 cm², 6,000 cm², 7,000 cm², 8,000 cm², 9,000 cm², or 10,000 cm²). In some embodiments the PHH are cultured on a two-dimensional surface, wherein the two-dimensional surface includes a surface area of between 636 cm² to 6360 cm².

### ECM

As described herein, the methods for culturing PHH may include culturing one or more PHH in contact with an ECM. In some embodiments, the PHH contact the ECM through physical, mechanism, or chemical means, or any combination thereof. Any suitable ECM may be used. Isolated PHH are preferably cultured in a microenvironment that mimics, at least in part, a cellular niche in which said PHH naturally reside. A cellular niche is determined in part by the PHH and surrounding cells, and the ECM that is produced by the cells in said niche. This cellular niche may be mimicked by culturing said PHH in the presence of biomaterials, such as an ECM that provides key regulatory signals controlling hepatocyte fate.

In some embodiments, the PHH adhere to the ECM. In some embodiments, the cell culture surface is coated with an ECM.

ECM includes a variety of polysaccharides, water, elastin, and glycoproteins, wherein the glycoproteins include collagen, entactin (nidogen), fibronectin, and laminin. ECM is secreted by connective tissue cells. Different types of ECM are known, including different compositions including different types of glycoproteins and/or different combination of glycoproteins. Said ECM can be provided by culturing ECM-producing cells, such as for example fibroblasts, in a receptacle, prior to the removal of these cells and the addition of isolated tissue fragments or isolated PHH. Examples of ECM-producing cells are chondrocytes, producing mainly collagen and proteoglycans, fibroblasts, producing mainly type IV collagen, laminin, interstitial procollagens, and fibronectin, and colonic myofibroblasts producing mainly collagens (type I, III, and V), chondroitin sulfate proteoglycan, hyaluronic acid, fibronectin, and tenascin-C.

Alternatively, ECM is commercially provided. Examples of commercially available ECMs are ECM proteins (Invitrogen). A synthetic ECM material may be used. Mixtures of ECM materials may be used, if desired. In some embodiments, the ECM does not include a hydrogel (e.g., MATRIGEL™). In some embodiments, the ECM includes a hydrogel (e.g., MATRIGEL™). In some embodiments, the hydrogel is MATRIGEL™.

In some embodiments, the ECM includes collagen. In some embodiments, the collagen is collagen-I or collagen-IV. In some embodiments, the collagen is collagen-I. In some embodiments, the collagen is collagen-IV.

In some embodiments, the ECM includes laminin. In some embodiments, the laminin is laminin-111, laminin-211, laminin-221, laminin-332, laminin-411, laminin-421, laminin-511, or laminin-521. In some embodiments, the laminin is laminin-111. In some embodiments, the laminin is laminin-511. In some embodiments, the laminin is laminin-521.

In some embodiments, the culture medium is placed on top of the ECM.

The culture medium can then be removed and replenished as and when required. In some embodiments, the culture medium is replenished every day. In some embodiments, the culture medium is replenished every alternate day. In some embodiments, the culture medium is replenished every third day.

In some embodiments, if components are “added” or “removed” from the media, then this can mean that the media itself is removed from the ECM and then a new media containing the “added” component or with the “removed” component excluded is placed on the ECM.

In some embodiments the culture medium of the invention is in contact with an ECM or a 3D matrix that mimics the ECM by its interaction with the cellular membrane proteins, such as integrins.

### Cellular Signatures

Albumin is a protein which is produced in the liver and prevents fluids from leaking out of the bloodstream. It is a hepatoblast and terminally differentiated hepatocyte marker. In some embodiments, after culturing, the PHH secrete albumin. Expanded PHH secrete lower levels of albumin as compared to overnight plated control PHH. In some embodiments, after maturation, the mature hepatocytes secrete albumin.

In some embodiments, the expanded PHH secrete at least 1 μg/million cells/day (e.g., at least 2 μg/million cells/day, 5 μg/million cells/day, 10 μg/million cells/day, 25 μg/million cells/day, or 50 μg/million cells/day) of albumin. For example, in some embodiments, the expanded PHH secrete at least 2 μg/million cells/day albumin. In some embodiments, the expanded PHH secrete at least 5 μg/million cells/day albumin. In some embodiments, the expanded PHH secrete at least 10 μg/million cells/day albumin. In some embodiments, the expanded PHH secrete at least 25 μg/million cells/day albumin. In some embodiments, the expanded PHH secrete at least 50 μg/million cells/day albumin.

It has been found that the self-renewing population of cells are those which are capable of expressing LGR5 on their surface. LGR5 positive cells proliferate by dividing to form clones, which further divide into clones and therefore expand the size of the cell population without the need for external intervention, without evolving into cells with a more restricted differentiation potential.

In some embodiments, the population of PHH that have been cultured in an expansion medium of the invention expresses the LGR5 cell surface marker.

In some embodiments, the LGR5 expression is induced in expanded PHH population whereas it is absent in overnight plated control PHH.

The liver breaks down proteins and produces nitrogen-containing ammonia. The nitrogen then combines with other elements, such as carbon, hydrogen, and oxygen, to form urea. In some embodiments, after culturing and/or expanding the PHH, the cells downregulate urea cycle enzyme transcript levels and secreted urea is not detected suggesting a need for an additional maturation step for expanded PHH to be able to secrete urea. In some embodiments, after maturation the mature hepatocytes secrete urea.

Adult mature PHH are also known to express one or more protein from the cytochrome p450 (CYP) protein family including Cyp3a4, Cyp1a2, nuclear receptor NR112, apical and basolateral polarity membrane proteins such as ABCG2, ABCC2, ABCB11, SR-B1, SLC10A1. In some embodiments, expanded PHH downregulate one or more of these proteins characteristic of mature cell state compared to unexpanded overnight plated control PHH. In some embodiments, expanded PHH upregulate one or more of fetal/hepatic progenitor/cholangiocyte markers such as AFP, Cyp3a7, EPCAM, LGR5, KRT7, KRT19, AQP1 compared to unexpanded overnight plated control PHH. In some embodiments, expanded PHH need additional maturation step to upregulate urea cycle enzymes, proteins characteristic of mature cell state and downregulate fetal/hepatic progenitor/cholangiocyte markers.

The presence and/or expression level/amount of various markers described herein in a sample can be analyzed by a number of methodologies, many of which are known in the art and understood by the skilled artisan, including, but not limited to, immunohistochemistry (“IHC”), Western blot analysis, immunoprecipitation, molecular binding assays, enzyme-linked immunosorbent assay (ELISA), enzyme-linked immuno-filtration assay (ELIFA), fluorescence activated cell sorting (“FACS”), MassARRAY, proteomics, quantitative blood based assays (e.g., Serum ELISA), biochemical enzymatic activity assays, in situ hybridization, fluorescence in situ hybridization (FISH), Southern analysis, Northern analysis, whole genome sequencing, massively parallel DNA sequencing (e.g., next-generation sequencing), NANOSTRING®, polymerase chain reaction (PCR) including quantitative real time PCR (qRT-PCR) and other amplification type detection methods, such as, for example, branched DNA, SISBA, TMA and the like, RNA-seq (bulk and single cell), microarray analysis, gene expression profiling, and/or serial analysis of gene expression (“SAGE”), as well as any one of the wide variety of assays that can be performed by protein, gene, and/or tissue array analysis. Typical protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis). Multiplexed immunoassays such as those available from Rules Based Medicine or Meso Scale Discovery (“MSD”) may also be used.

In some embodiments of any of the methods described herein, DNA from PHH can be isolated and subsequently sequenced using a next-generation sequencing method, such as the targeted gene pulldown and sequencing method described in Frampton et al. (Nature Biotechnology. 31: 1023-1033, 2013), which is incorporated by reference herein in its entirety.

In any of the preceding methods, the presence and/or expression level/amount of a marker (e.g., LGR5) is measured by determining protein expression levels of the marker. In some embodiments, the method includes contacting the biological sample with antibodies that specifically bind to a marker (e.g., anti-LGR5 antibodies) under conditions permissive for binding of the marker, and detecting whether a complex is formed between the antibodies and marker. Such method may be an in vitro or in vivo method. Any method of measuring protein expression levels known in the art or provided herein may be used. For example, in some embodiments, a protein expression level of a marker is determined using a method selected from the group consisting of flow cytometry (e.g., fluorescence-activated cell sorting (FACS™)), western blot, enzyme-linked immunosorbent assay (ELISA), immunoprecipitation, immunohistochemistry (IHC), immunofluorescence, radioimmunoassay, dot blotting, immunodetection methods, HPLC, surface plasmon resonance, optical spectroscopy, mass spectrometry, and HPLC.

In some embodiments, the presence and/or expression level/amount of a marker (e.g., LGR5) is measured by determining mRNA expression levels of the marker. In some embodiments, the expression level of a gene is determined using a method including: (a) performing gene expression profiling, PCR (such as RT-PCR), RNA-seq, microarray analysis, SAGE, MassARRAY technique, or FISH on a sample (such as a subject liver disease sample); and b) determining presence and/or expression level/amount of a marker in the sample. In one embodiment, the PCR method is qRT-PCR. In one embodiment, the PCR method is multiplex-PCR. In some embodiments, gene expression is measured by microarray. In some embodiments, gene expression is measured by qRT-PCR. In some embodiments, expression is measured by multiplex-PCR.

Methods for the evaluation of mRNAs in cells are well known and include, for example, hybridization assays using complementary DNA probes (such as in situ hybridization using labeled riboprobes specific for the one or more genes, Northern blot and related techniques) and various nucleic acid amplification assays (such as RT-PCR using complementary primers specific for one or more of the genes, and other amplification type detection methods, such as, for example, branched DNA, SISBA, TMA and the like). Samples from mammals can be conveniently assayed for mRNAs using Northern, dot blot, or PCR analysis. In addition, such methods can include one or more steps that allow one to determine the levels of target mRNA in a biological sample (e.g., by simultaneously examining the levels a comparative control mRNA sequence of a “housekeeping” gene such as an actin family member).

Additionally or alternatively to mRNA expression analysis, other markers, such as protein expression, may be quantified according to methods described above. For example, methods of the invention include testing a sample for a genomic marker (e.g., the presence of AFP) and additionally testing a sample for a protein marker (e.g., protein transcripts of AFP).

In some embodiments of any of the methods, a DNA sequence may serve as a marker. DNA can be quantified according to any method known in the art, including, but not limited to, PCR, exome-seq (e.g., whole exome sequencing), DNA microarray analysis, NANOSTRING®, or whole genome sequencing.

In some instances, the expression level of the genes in the sample is an average (e.g., mean expression or median expression) of the genes, the reference expression level of the genes is an average (e.g., mean expression or median expression) of the genes of the reference, and the average of the genes of the sample is compared to the average of the genes of the reference.

In some embodiments, the presence and/or expression levels/amount of a marker in a first sample is increased or elevated as compared to presence/absence and/or expression levels/amount in a second sample. In some embodiments, the presence/absence and/or expression levels/amount of a marker in a first sample is decreased or reduced as compared to presence and/or expression levels/amount in a second sample. In some embodiments, the second sample is a reference sample (e.g., PHH cultured in the absence of the disclosed, defined culture media), reference cell, reference tissue, control sample, control cell, or control tissue.

### Vessels and Incubators

The disclosure also features tissue cultures and incubators that may be used with the media and methods described herein. For example, the culture method described herein may include culturing PHH in a vessel or incubator.

In some embodiments, the tissue culture vessel may include the expansion medium described herein. In some embodiments, an incubator may include the tissue culture vessel. In some embodiments, the incubator may include the tissue culture vessel and maintain the tissue culture vessel under hypoxic conditions. Hypoxic conditions may include, e.g., an oxygen level of less than 20%. In some embodiments, the incubator maintains an oxygen level of between 1% to 19% (e.g., between 1% and 10%, e.g., 1%, 2%, 3%, 4%, 5%, 6%, 7%, 8%, 9%, 10%, 11%, 12%, 13%, 14%, 15%, 16%, 17%, 18%, 19%, or 20%). In some embodiments, the oxygen level is between 1% to 10%. In some embodiments, the oxygen level is 5%. In some embodiments, an incubator may include the tissue culture vessel and maintain the tissue culture vessel under normoxic conditions.

### Hepatocyte Aggregates

The expanded and/or matured PHH of the invention may be aggregated. Aggregates described herein include a population of PHH. In some embodiments, the PHH are admixed under conditions which cause the cell population to form aggregates. In some embodiments, the PHH are admixed using tissue fabrication techniques. In some embodiments, the PHH are cultured by hanging drop, microwell molding, non-adhesive surfaces, spheroid suspension culture using a spinner flask, vertical wheel bioreactor, horizontal wheel bioreactor, or a microfluidic spheroid system. Additional methods include those using acoustical waves and using positively-charged surfaces on a plate. In some embodiments, the PHH are admixed in the presence of stromal cells (e.g., normal human dermal fibroblasts (NHDF)). In some embodiments, the PHH are admixed in the presence of NHDF. In some embodiments, the PHH are admixed in the absence of stromal cells (e.g., NHDF). In some embodiments, the PHH are admixed in the absence of NHDF.

In other aspects, the compositions provided herein can contain additional components, including but not limited to, growth factors, ligands, cytokines, drugs, etc. In some embodiments, the cell mixture can include molecules which elicit additional microenvironmental cues such as small molecules or growth factors which stimulate or enhance proliferation and expansion of a cell population.

In certain embodiments, the aggregates disclosed herein include one or more adherence materials to facilitate maintenance of the desired phenotype of the grafted cells in vivo. The material may include, but is not limited to, antibodies, proteins, peptides, nucleic acids, peptide aptamers, nucleic acid aptamers, sugars, proteoglycans, or cellular receptors. The type of adherence materials (e.g., ECM materials, sugars, proteoglycans, etc.) will be determined, in part, by the cell type (e.g., PHH) to be cultured.

In some embodiments, organizing cells and material into spatial arrangements, such as aggregates, can be accomplished by physically constraining the placement of cells/material by the use of wells or grooves, or injecting cells into microfluidic channels or oriented void spaces/pores. In certain embodiments, the cells can be organized by physically positioning cells with electric fields, magnetic tweezers, optical tweezers, ultrasound waves, pressure waves, or micromanipulators.

The cells produced by the methods described herein can be used immediately in the making of an aggregate. Alternatively, the cells can be frozen at liquid nitrogen temperatures and stored for long periods of time, being thawed and capable of being reused. For example, the cells can be frozen in 10% DMSO, 50% serum, 40% buffered medium, or some other such solution as is commonly used in the art to preserve cells at such freezing temperatures and thawed in a manner as commonly known in the art for thawing frozen cultured cells.

### Cell Implants

As described herein, the methods disclosed herein may include introducing the population of expanded and/or matured PHH or progeny thereof into a recipient. In some embodiments, the population of expanded and/or matured PHH or progeny thereof is introduced into a recipient in the form of a hepatocyte aggregate. In some embodiments, the recipient is a human. In some embodiments, the recipient is a human patient suffering from a liver disease. The population of expanded and/or matured PHH or progeny thereof may be incorporated into an engineered tissue construct, e.g., for implantation into a subject. The engineered tissue construct may include a biocompatible hydrogel scaffold (e.g., containing fibrin). The biocompatible scaffold may contain an encapsulated population of aggregated PHH.

In some embodiments, uses of the population of expanded and/or matured PHH cultured as described herein are likewise provided. For example, in some embodiments, the invention also provides the use of the population of expanded and/or matured PHH of the invention or a lot of frozen PHH derived from said population of expanded PHH in a discovery screen; toxicity assay; gene expression studies including recombinant gene expression; research of mechanisms involved in tissue injury and repair; research of inflammatory and infectious diseases; studies of pathogenetic mechanisms; or studies of mechanisms of cell transformation and etiology of liver disease.

In some embodiments, the invention also provides cells derived from the population of expanded and/or matured PHH of the invention for use in medicine. In some embodiments, the invention also provides cells derived from the population of expanded and/or matured PHH of the invention for use in treating a disorder, condition, or disease. In some embodiments, the invention also provides the population of expanded PHH, or cells derived from the population of expanded PHH of the invention, for use in regenerative medicine, for example, wherein the use involves implantation of the population of expanded cells or cells derived from the population of expanded PHH into a patient. In some embodiments, the invention also provides the population of matured PHH, or cells derived from the population of matured PHH of the invention, for use in regenerative medicine, for example, wherein the use involves implantation of the population of matured cells or cells derived from the population of matured PHH into a patient.

### Pharmaceutical Formulations

The invention also provides a pharmaceutical formulation including one or more population of expanded and/or matured PHH and a pharmaceutically acceptable diluent and/or excipient. In some embodiments, pharmaceutical formulations include a population of PHH and one or more excipients. In some embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In some embodiments, expansion media or maturation media may be admixed with pharmaceutically acceptable active and/or inert substances for the preparation of pharmaceutical compositions or formulations. Compositions and methods for the pharmaceutical formulations depend on a number of criteria, including, but not limited to, route of administration, extent of disease, or dose to be administered.

In some embodiments, pharmaceutical compositions including a population of PHH encompass any pharmaceutically acceptable salts of the inhibitor, esters of the inhibitor, or salts of such esters. In some embodiments, pharmaceutical compositions including a population of PHH, upon administration to a subject (e.g., a human), are capable of providing (directly or indirectly) the biologically active metabolite or residue thereof. Accordingly, for example, the disclosure is also drawn to pharmaceutically acceptable salts of inhibitors, prodrugs, pharmaceutically acceptable salts of such prodrugs, and other bioequivalents. Suitable pharmaceutically acceptable salts include, but are not limited to, sodium and potassium salts. In some embodiments, prodrugs include one or more conjugate group attached to an expansion medium, wherein the conjugate group is cleaved by endogenous nucleases within the body.

In some embodiments, pharmaceutical formulations include a co-solvent system. Certain of such co-solvent systems include, for example, benzyl alcohol, a nonpolar surfactant, a water-miscible organic polymer, and an aqueous phase. In some embodiments, such co-solvent systems are used for hydrophobic compounds. A non-limiting example of such a co-solvent system is the VPD co-solvent system, which is a solution of absolute ethanol including 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In some embodiments, a pharmaceutical formulation is prepared for administration by injection (e.g., intraocular (e.g., intravitreal), intravenous, subcutaneous, intramuscular, intrathecal, intracerebroventricular, etc.). In some embodiments, a pharmaceutical formulation includes a carrier and is formulated in aqueous solution, such as water or physiologically compatible buffers such as Hanks's solution, Ringer's solution, or physiological saline buffer. In some embodiments, other ingredients are included (e.g., ingredients that aid in solubility or serve as preservatives). In some embodiments, injectable suspensions are prepared using appropriate liquid carriers, suspending agents and the like.

Certain pharmaceutical formulations for injection are presented in unit dosage form, e.g., in ampoules or in multi-dose containers. Certain pharmaceutical formulations for injection are suspensions, solutions, or emulsions in oily or aqueous vehicles, and may contain formulatory agents such as suspending, stabilizing and/or dispersing agents. Certain solvents suitable for use in pharmaceutical formulations for injection include, but are not limited to, lipophilic solvents and fatty oils, such as sesame oil, synthetic fatty acid esters, such as ethyl oleate or triglycerides, and liposomes.

### Kits

The compositions described herein can be provided in a kit for use in expanding freshly extracted or previously frozen PHH or for maturing PHH. In some embodiments, the kit can include one or more cell culture medium component as described herein. In some embodiments, the kit can include a package insert that instructs a user of the kit, such as a laboratory scientist, to perform any one of the methods described herein. In some embodiments, the kit can include frozen PHH that were previously expanded according to the method described herein. In some embodiments, the kit can include frozen PHH that were previously matured according to a method described herein. In some embodiments, the kit can optionally include equipment for administering the pharmaceutical formulation.

### EXAMPLES

The following examples are put forth so as to provide those of ordinary skill in the art with a description of how the compositions and methods described herein may be used and evaluated and are intended to be purely exemplary of the invention and are not intended to limit the scope of what the inventors regard as their invention.

### Example 1. Evaluation of Cell Culture Method Using Expansion Medium

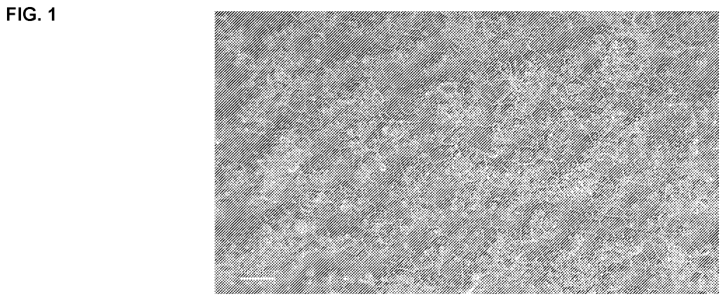

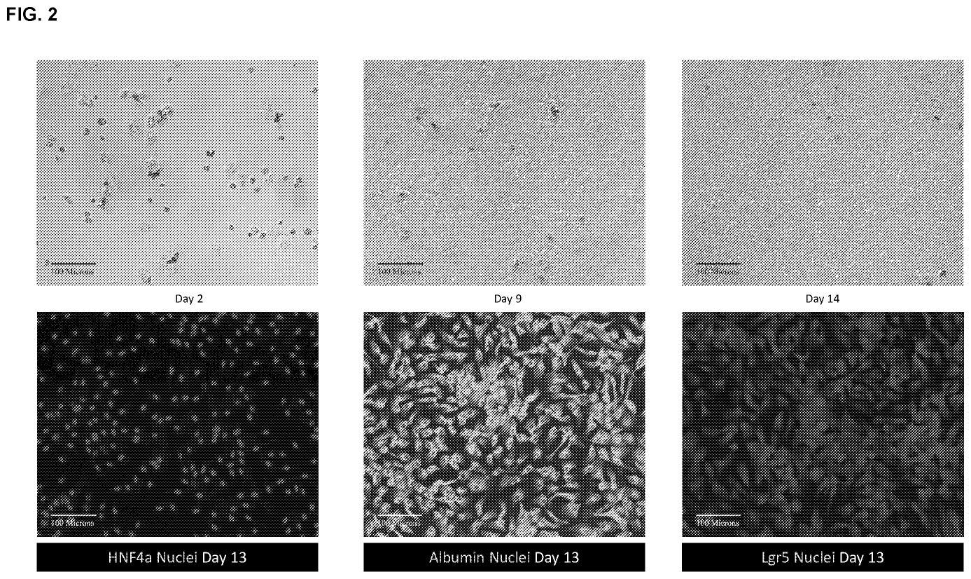

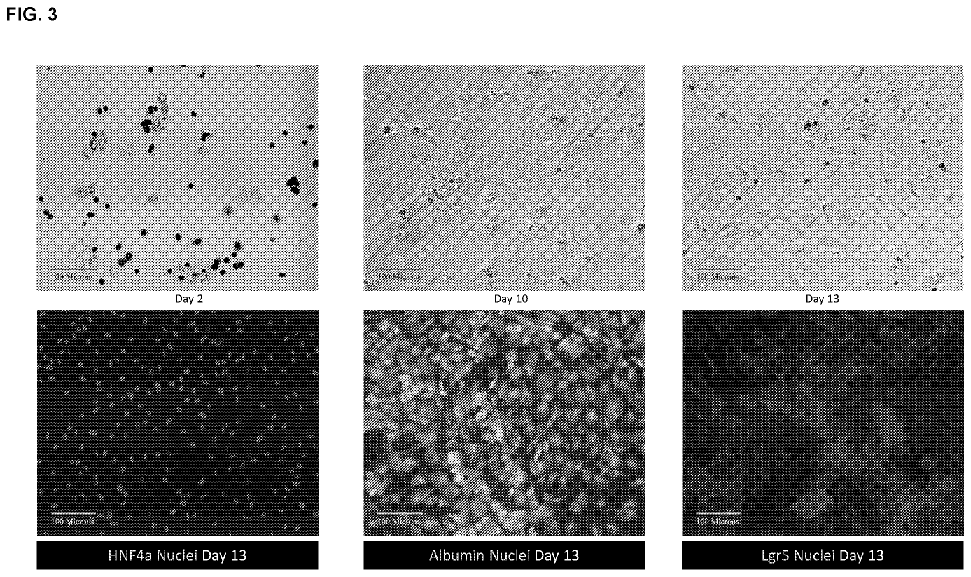

Frozen vials of primary human hepatocytes (PHH) were thawed according to vendor-provided instructions. PHH were plated on rat tail collagen-I or recombinant human laminin-521 coated tissue-culture vessels at a density of 5×10³ to 6×10³ per cm² in the presence of the vendor-provided medium (Table 1, Table 2). After four hours, plating media was replaced with the disclosed expansion medium, which was replaced every 48 hours. FIG. 1 shows a brightfield image of PHH cells cultured using the expansion medium at (P0) after 6 days in a T-75 culture flask. PHH were collected at different time points and immunofluorescence was performed by fixing PHH in 4% formaldehyde, permeabilizing PHH in 0.4% Triton X-100, blocking in bovine serum albumin, incubating overnight with a primary antibody selected from albumin, HNF4α, and LGR5, and incubating for one hour with the appropriate secondary antibody. Images were taken on a fluorescence microscope. After 13 days of growth on a two-dimensional plastic surface coated with collagen-I, PHH expressed albumin, HNF4α, and LGR5 (FIG. 2). After 13 days of growth on a two-dimensional plastic surface coated with laminin-521, PHH expressed albumin, HNF4α, and LGR5 (FIG. 3). Qualitative comparisons between the immunostained cell panels demonstrate that collagen-I and laminin-521 yield comparable staining results. The use of an ECM for culturing PHH enhanced long-term survival of the PHH and the continued presence of the proliferative hepatic phenotype.

TABLE 1
Processes and pathways targeted by expansion medium components
Processes and Pathways

| | |
|---|---|
| 1 | Activation of Wnt Signaling |
| 2 | Liver Regeneration Activators |
| 3 | Stabilization of Epithelial Phenotype |
| 4 | Promotion of Cell Survival and Proliferation |

TABLE 2
Expansion medium (Expand 1.0) composition

| | | EXPAND | | Processes and |
|---|---|---|---|---|
| | Components | Cocktail | | Pathways Targeted |
| 1 | Advanced DMEM/F-12 | Base Medium | | |
| 2 | Penicillin/Streptomycin | 1% | | |
| 3 | GLUTAMAX ™ | 1% | | |
| 4 | HEPES | 10 | mM | |
| 5 | B27 Supplement (50X), | 1x | | 4 |
| | without Vitamin A | | | |
| 6 | N2 Supplement (100X) | 1x | | 4 |
| 7 | Rspo-1 | 100 | ng/mL | 1 |
| 8 | Wnt3a | 50 | ng/mL | 1 |
| 9 | N-acetylcysteine | 1.25 | mM | 4 |
| 10 | Nicotinamide | 100 | ng/mL | 4 |
| 11 | Recombinant EGF | 50 | ng/mL | 2, 4 |
| 12 | Recombinant TGFα | 20 | ng/mL | 2 |
| 13 | Recombinant human FGF-7 | 50 | ng/mL | 4 |
| 14 | Recombinant human FGF-10 | 50 | ng/mL | 4 |
| 15 | Recombinant human HGF | 25 | ng/mL | 2, 4 |
| 16 | A83-01 | 2 | μM | 3 |
| 17 | ROCK Inhibitor (Y-27632) | 10 | μM | 4 |

### Example 2. Evaluation of Expansion of PHH

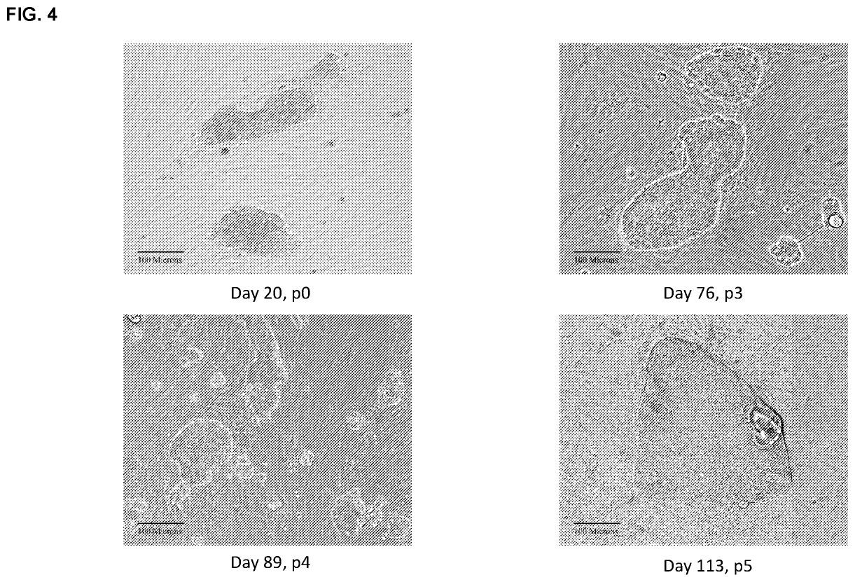

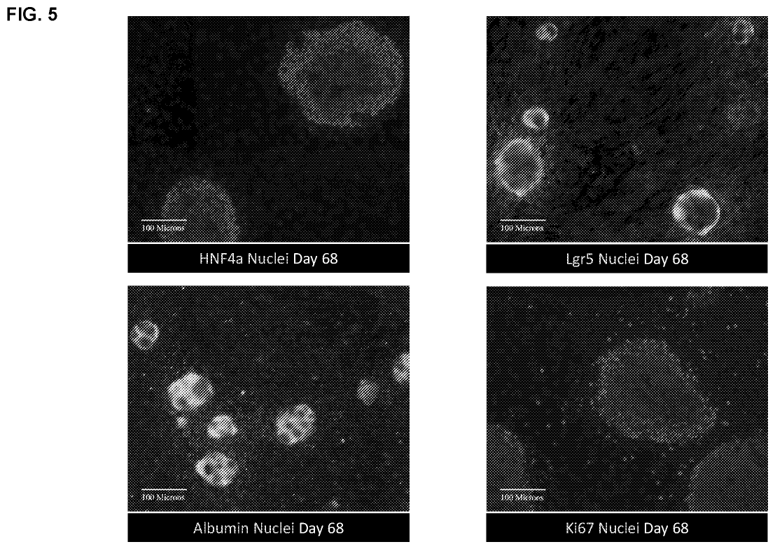

PHH were passaged and expanded for 120 days on a two-dimensional surface coated with laminin-521 in the presence of the expansion medium. The medium was refreshed every 48 hours. During this period, the cells maintained their morphology and epithelial phenotype as seen in (FIG. 4). On day 68, immunostaining of the cells revealed that the cells on the leading edge of the colonies were proliferative hepatocyte phenotype cells which expressed HNF4α, LGR5, and Ki67 (FIG. 5).

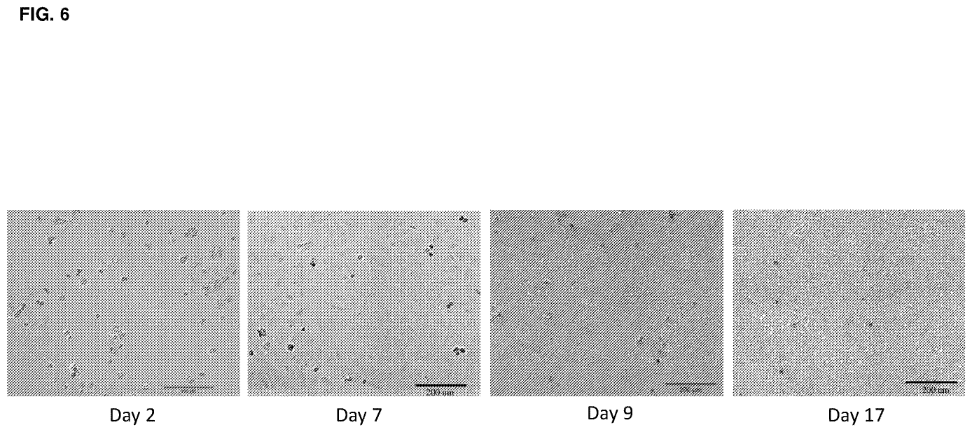

The panels in FIG. 6 represent PHH cultured in expansion medium described in Table 2 on day 2, day 7, day 9, and day 17, respectively. By day 2, approximately 20-25% of the seeded cells appear to have adhered to the cell culture surface. By day 7, the cells are actively dividing and have increased in size. At day 9, the cells are fully confluent and display the cobble stone pattern that is a hallmark of the epithelial phenotype. On day 17, the cells exhibit well-defined hepatocyte morphology and are tightly packed. In comparison to earlier time points, their size appears smaller.

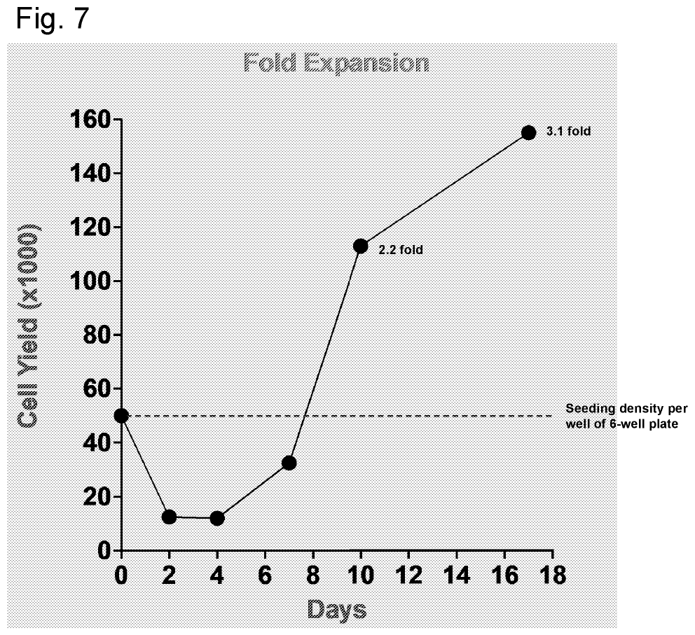

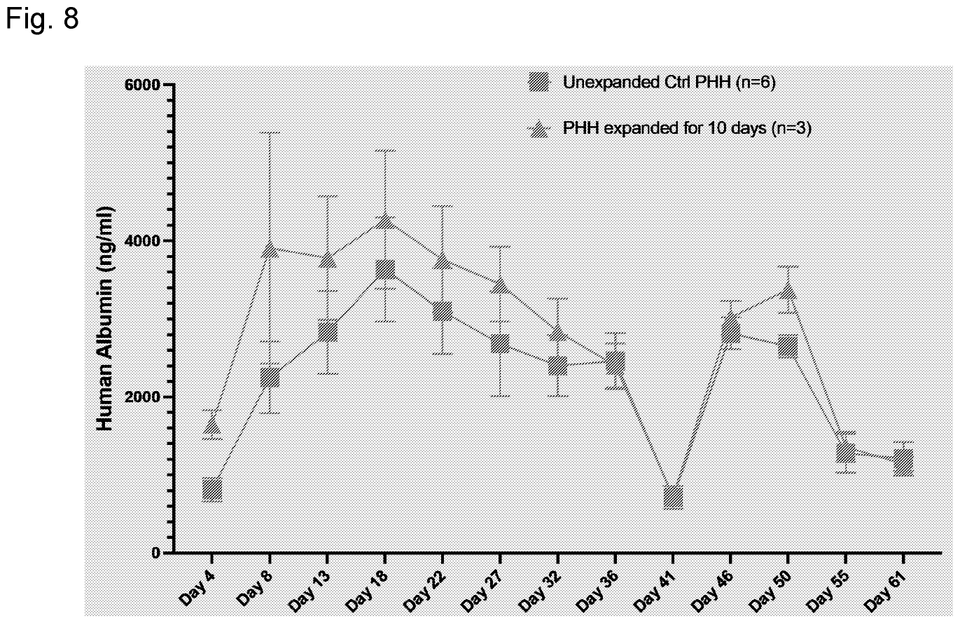

PHH (50×10³ cells per well) were seeded onto the surface of 6-well tissue culture plate and cultured in the presence of the expansion medium (FIG. 7, Day 1). By day 17, the cells expanded 3.1-fold such that one well of a 6-well plate contained 15.5×10⁴ cells per well (FIG. 7, Day 17) (FIG. 8).

### Example 3. Expanded Cells Implanted into a Mouse

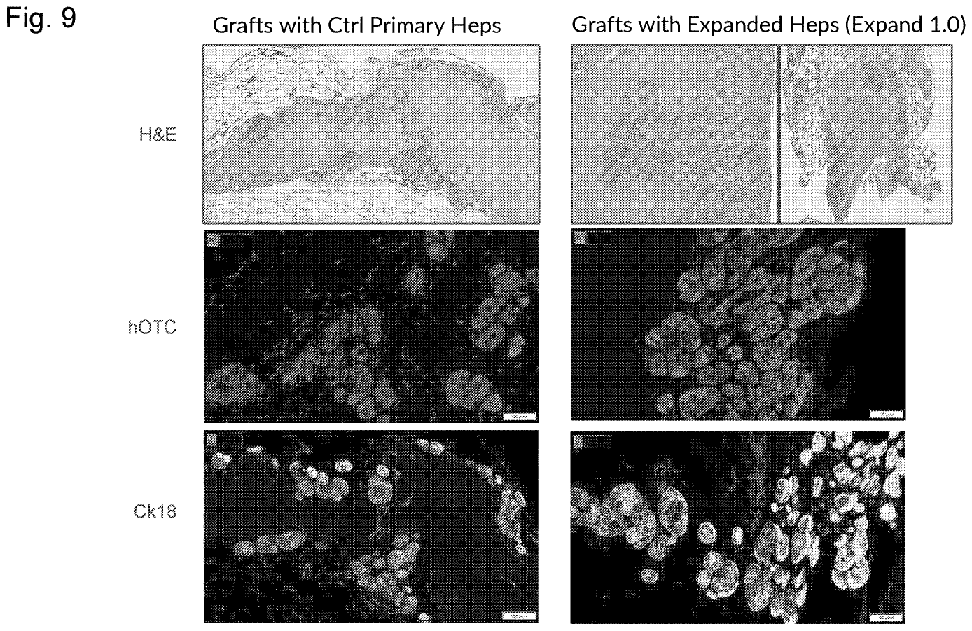

PHH (4.5×10⁵ cells) were aggregated with 9×10⁵ normal human dermal fibroblasts (NHDF) in 7 mg/mL bovine fibrinogen to form one cell implant. PHH lot D listed in Table 3 was used in this experiment. Unexpanded PHH and PHH expanded on Collagen-I matrix for 10 days using expansion medium described in FIG. 1 were used to prepare grafts for implantation. Three NOD-scid IL2Rgamma^{null} (NSG™) mice (5-8 weeks of age upon arrival) received one implant each of day 10 expanded PHH aggregates, and six NSG™ mice received one implant each of unexpanded PHH aggregates in control group. NSG™ mice are immunodeficient mice. For two months, longitudinal clinical observation was conducted primarily by three blood draws every two weeks to detect and quantify secreted human albumin levels by ELISA (FIG. 8). The detection of human albumin in mice transplanted with these aggregates demonstrated that the PHH in the aggregates were viable. Furthermore, it confirmed that the expanded PHH were healthy, as they were able to continuously synthesize and secrete human albumin two months after implantation. Terminal analysis of expanded grafts was also performed by immunohistochemistry to confirm the presence of hepatocytes in the grafts. Immunohistochemistry confirmed presence of healthy hepatic aggregates by H&E, hOTC, and CK18 staining (FIG. 9).

TABLE 3
Donor lots of PHH

| | | | | | Expansion | |
|---|---|---|---|---|---|---|
| PHH | | | | Social History/ | (Quali- | Seeding |
| Lot | Age | | Gender | Medication Use | tative) | Density |
| A | 50 | Years | Male | Marijuana, | + | 40k/well |
| | | | | cocaine | | |
| B | 6 | Months | Male | Albuterol, | ++ | 100k/well |
| | | | | Sildenafil | | |
| C | 3 | Years | Male | Baclofen, | ++ | 100k/well |
| | | | | Valium, | | |
| | | | | Trileptal, | | |
| | | | | Zyrtec | | |
| D | 14 | years | Female | N/A | +++++ | 50k/well |

### Example 4. Evaluation of Expansion Limit of PHH

PHH were passaged and expanded on a two-dimensional surface coated with an ECM including collagen-I and laminin-511 in the presence of the expansion medium. The expansion medium included Wnt signaling activators Wnt3a and roof plate-specific spondin protein 4, receptor tyrosine kinase ligands epidermal growth factor and transforming growth factor-alpha, epithelial phenotype stabilizing agent A83-01, and cell survival agents nicotinamide and B27. The medium was refreshed every alternate day, and

PHH were passaged upon reaching confluence. PHH were seeded onto a new surface at 10-fold dilution. After six months of culturing, a sample of PHH was collected. Immunofluorescence was performed to evaluate the PHH expression profile. Immunofluorescence quantification showed that at most 15% (e.g., at most 10%, 5%, or 1%) of the PHH expressed Ki67, at least 10% (e.g., at least 11%, 12%, 13%, 14%, or 15%) of the cells expressed Cyp3a4, and at least 80% (e.g., at least 85%, 90%, or 95%) of the cells expressed one or more protein selected from hepatocyte nuclear factor 4 alpha, leucine rich repeat containing G protein-coupled receptor 5, keratin 18, and albumin.

### Example 5. Expansion of PHH Using Modified Expansion Medium—EXPAND 3.0 Cocktail

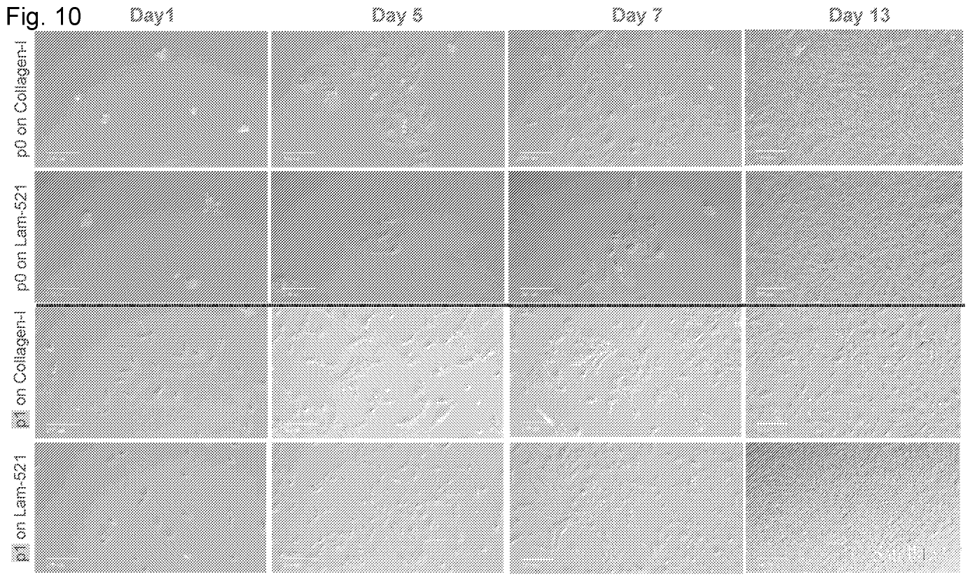

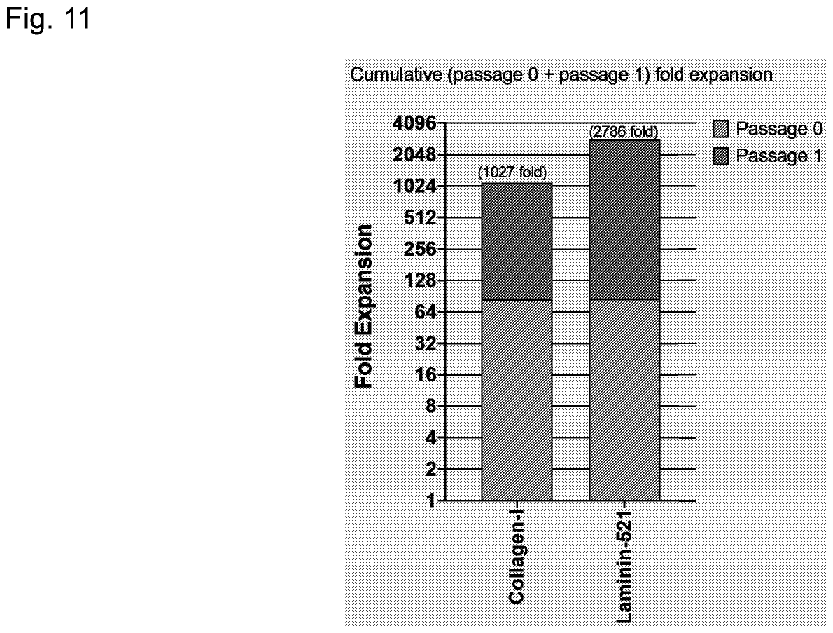

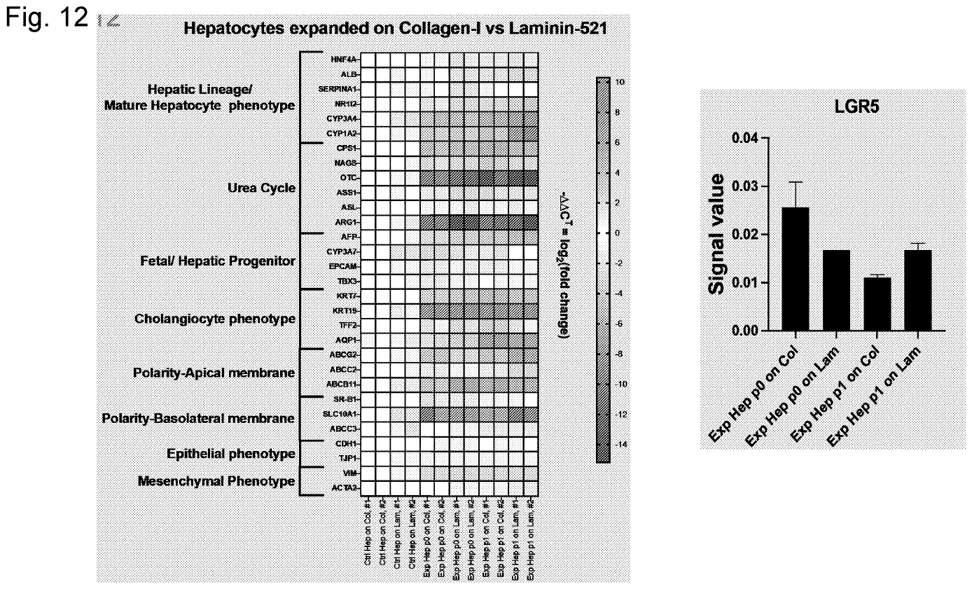

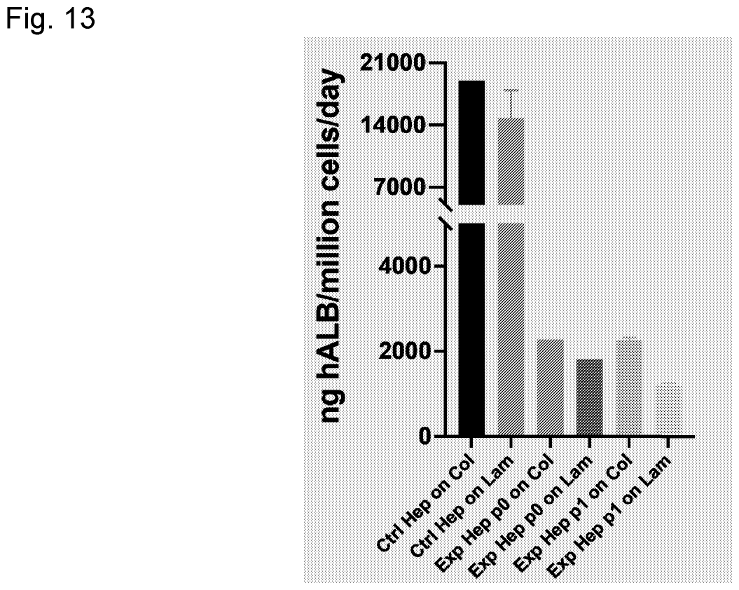

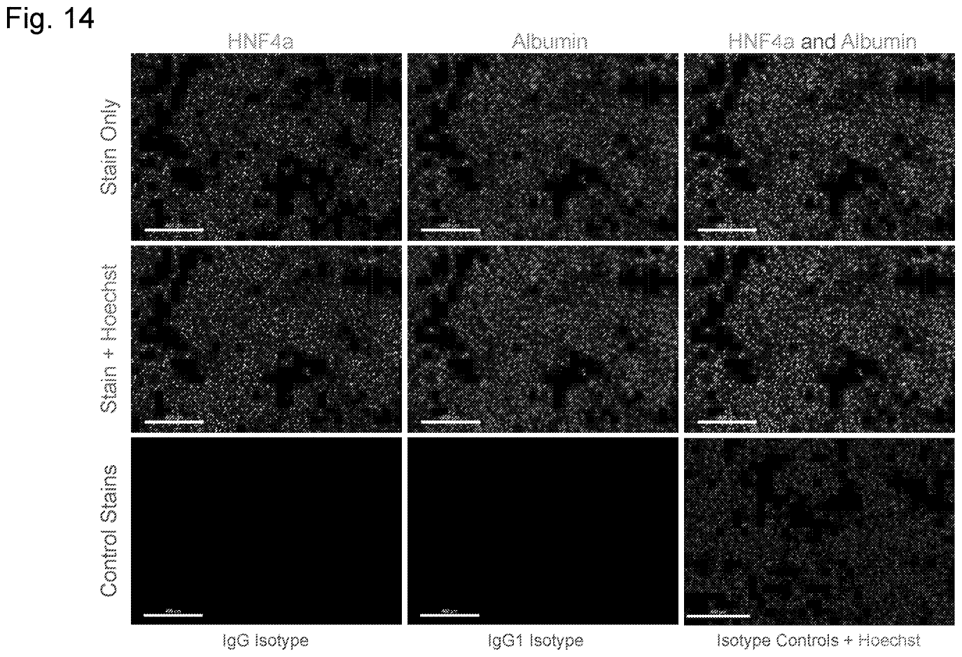

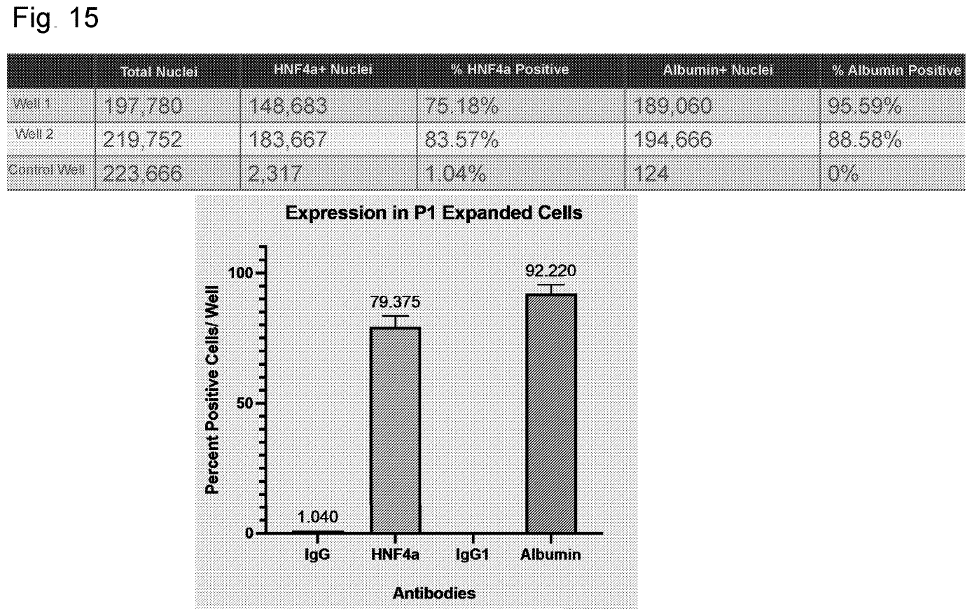

PHH were expanded in a similar manner as described in Example 1 using a modified expansion medium, EXPAND 3.0 Cocktail. The modified expansion media lacked ROCK inhibitor and included KNOCKOUT™ serum replacement (KOSR) and nonessential amino acids (NEAA) (Table 4). KOSR was added at a gradient increase of 5-10% for EXPAND 3.0 Cocktail. The expanding PHH were placed in an incubator with a 5% oxygen hypoxic atmosphere for Expand 3.0 Cocktail. These changes to culture conditions resulted in robust expansion of PHH on both Collagen-I and Laminin-521 matrix (FIG. 10). Upon further optimization of passaging and P1 expansion, Expand 3.0 supported >1000 fold cumulative expansion in P0+P1 (FIG. 11). RT-qPCR analysis of expanded hepatocytes in P0 and P1 demonstrated hepatic progenitor cell state of expanded hepatocytes compared to control unexpanded hepatocytes that are plated overnight on Collagen-I or Laminin-521 (FIG. 12). Less mature cell state of expanded hepatocytes compared to control unexpanded hepatocytes is demonstrated by lower level of secreted albumin (FIG. 13). Hepatic phenotype of expanded hepatocytes at the end of p1 expansion is confirmed immunostaining for HNF4a and Albumin (FIG. 14) and quantifying percentage positive cells (FIG. 15).

TABLE 4
Modified expansion medium compositions

| | Components | EXPAND 3.0 Cocktail | |
|---|---|---|---|
| 1 | Advanced DMEM/F-12 | Base Medium | |
| 2 | Penicillin/Streptomycin | 1% | |
| 3 | GLUTAMAX ™ | 1% | |
| 4 | HEPES | 10 | mM |
| 5 | B27 Supplement (50X), | 1x | |
| | without Vitamin A | | |
| 6 | N2 Supplement (100X) | 1x | |
| 7 | Rspo-1 | 100 | ng/mL |
| 8 | Wnt3a | 50 | ng/mL |
| 9 | N-acetylcysteine | 1.25 | mM |
| 10 | Nicotinamide | 100 | ng/mL |
| 11 | Recombinant EGF | 50 | ng/mL |
| 12 | Recombinant TGFα | 20 | ng/mL |
| 13 | Recombinant human FGF-7 | 50 | ng/mL |
| 14 | Recombinant human FGF-10 | 50 | ng/mL |
| 15 | Recombinant human HGF | 25 | ng/mL |
| 16 | A83-01 | 2 | μM |

| | | |
|---|---|---|
| 17 | KOSR | 1-5-10% or 5-10% |
| 18 | NEAA | 1X |
| | Hypoxic culture conditions? | Yes |

### Example 6. Maturation of Expanded Hepatocytes

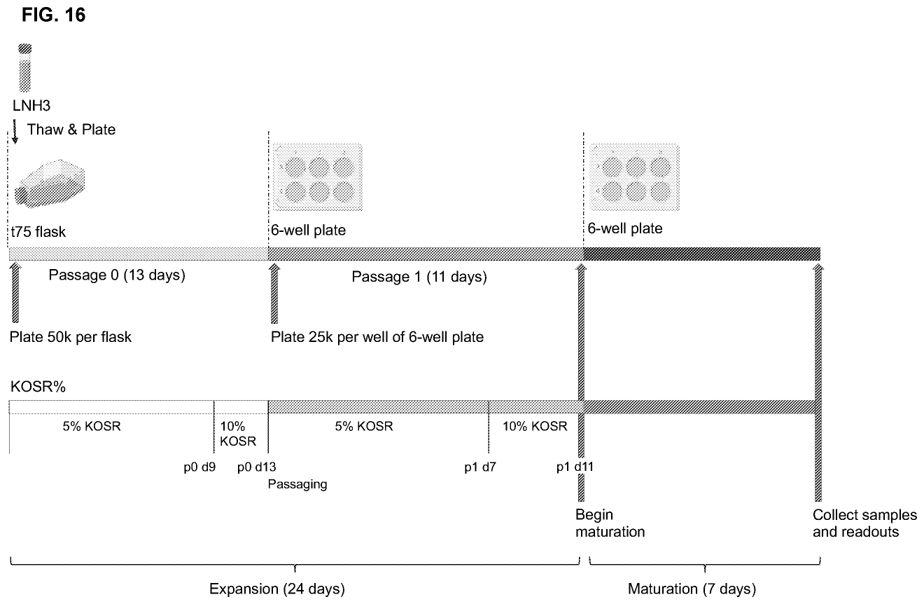

PHH were expanded in a similar manner as described in Examples 1 and 5 and then were allowed to undergo maturation (FIG. 16). Frozen vials of PHH were thawed and plated into a t75 flask with expansion media containing 5% KOSR (step P0). After 9 days, KOSR was raised to 10%. On day 13, cells were moved into a 6-well plate (step P1) with fresh media containing 5% KOSR and left for 7 days before the KOSR was raised to 10% and the cells were allowed to expand for 4 more days. The total cell expansion time included 24 days. After expansion, cells were moved into a 6-well plate containing a maturation base media of LONZA™ HCM™, William's E, or Hepatozyme-SFM with various hepatocyte maturation supplements (Table 5).

TABLE 5
Supplements used for maturing expanded hepatocytes

| | | | | Selected function relevant to |
|---|---|---|---|---|
| Supplement | | Concentration | | hepatocyte formation |
| 1 | Penicillin/Streptomycin | 1x | | |
| | (100x) | | | |
| 2 | HEPES (100x) | 1x | | |
| 3 | GLUTAMAX ™ (100x) | 1x | | |
| 4 | Insulin Transferrin | 1x | | |
| | Selenium (ITS) (100x) | | | |
| 5 | Compound E | 0.1 | uM | Notch inhibitor to prevent cholangiocyte |
| | | | | differentiation of expanded hepatocytes |
| 6 | Erlotinib HCl | 2 | uM | EGFR inhibitor to prevent cholangiocyte |
| | | | | differentiation |
| 7 | Oncostatin M | 20 | ng/ml | Induces hepatocyte maturation |
| 8 | Dexamethasone | 10 | uM | Glucocorticoid, induces hepatocyte |
| | | | | maturation |
| 9 | Hydrocortisone (Lonza | 1x | | Glucocorticoid, induces hepatocyte |
| | CC-4182, 1000x) | | | maturation |
| 10 | Vitamin C | 0.5 | mM | Anti-oxidant, to improve overall cell health |
| 11 | Vitamin K2 | 10 | uM | Activates PXR, which is necessary for |
| | | | | expression of CYP enzymes |
| 12 | Lithocholic Acid | 10 | uM | Secondary bile acid, activates PXR. Bile |
| | | | | acids are important for hepatocyte polarity. |
| 13 | Urso deoxycholic Acid | 25 | uM | Secondary bile acid, shown to improve |
| | | | | liver function. Bile acids are important for |
| | | | | hepatocyte polarity. |
| 14 | 8 Bromo cAMP | 1 | mM | Induces expression of PGC1a which works |
| | | | | with HNF4a to activate hepatic gene |
| | | | | expression |
| 15 | Forskolin | 5 | uM | cAMP analog |
| 16 | Cholesterol (250x) | 1x | | Necessary for cell membrane integrity. It |
| | | | | is also needed for bile acid synthesis. Bile |
| | | | | acids promote hepatocyte polarity. |
| 17 | T3 | 3 | uM | Increases expression of CYP3A4 and |

decreases AFP expression

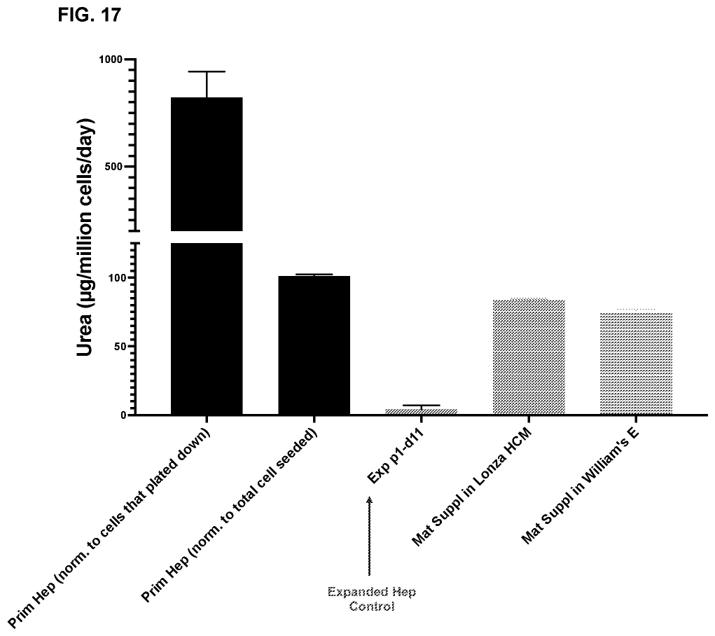

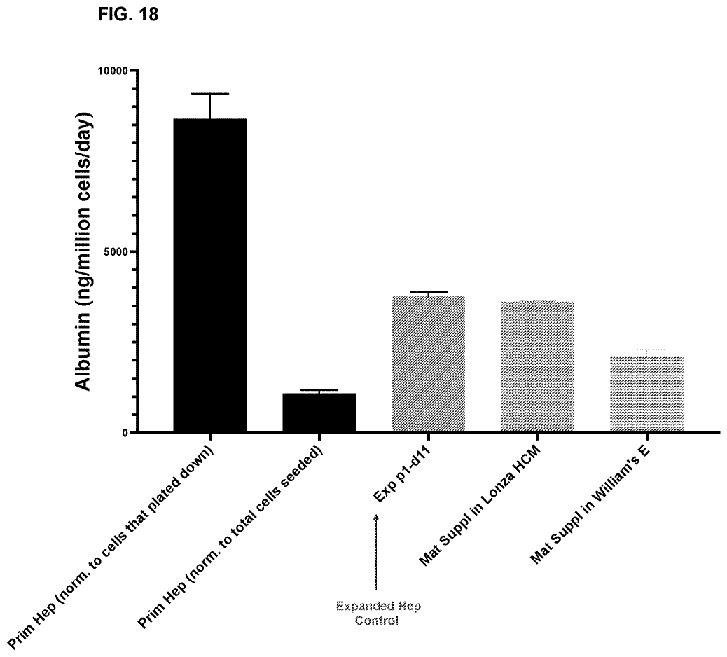

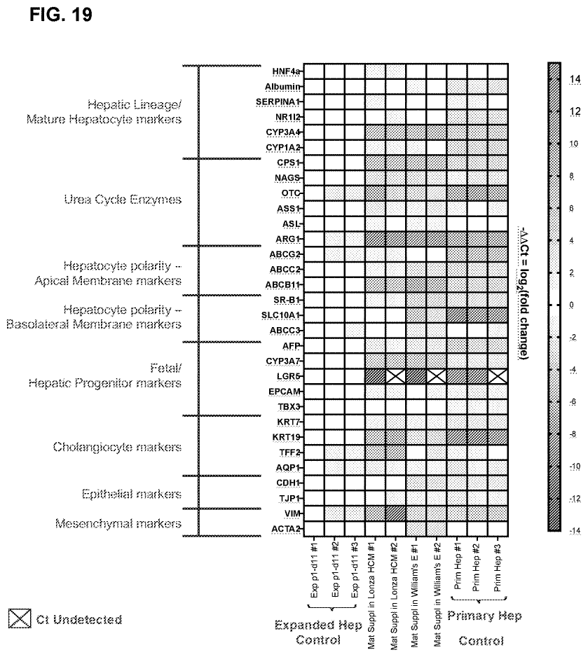

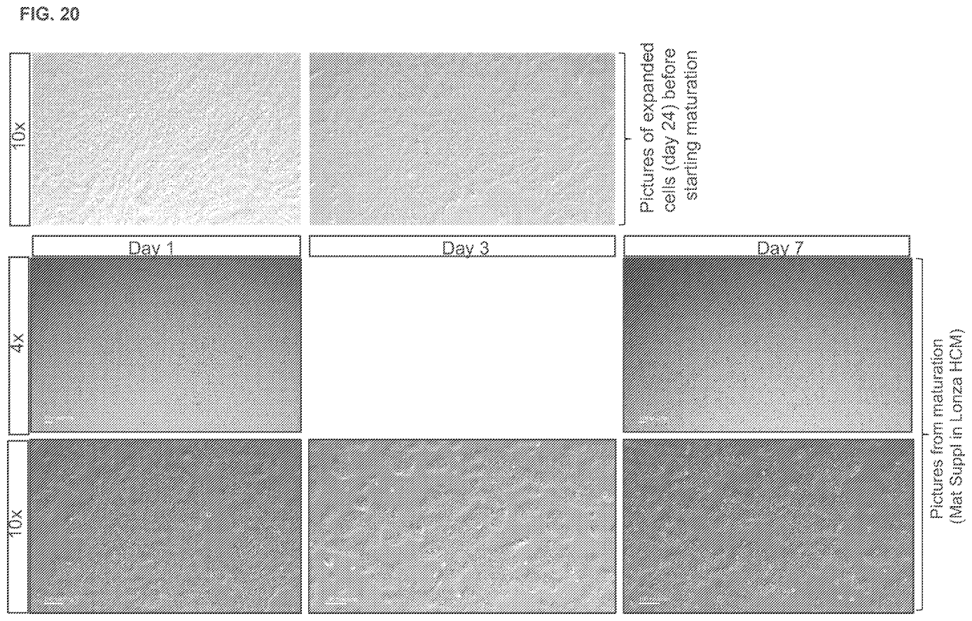

After 7 days of maturation, samples were collected and urea, albumin, and maturation markers were measured. Maturation of expanded hepatocytes resulted in the induction and secretion of urea (FIG. 17), while levels of secreted albumin were maintained (FIG. 18). Transcripts corresponding to the mature hepatocyte phenotype were found to be upregulated upon maturation of expanded hepatocytes while progenitor/cholangiocyte transcripts were downregulated (FIG. 19). Phase contrast microscopy images showed morphological differences between expanded hepatocytes and hepatocytes after completion of maturation (FIG. 20).

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A method for culturing primary human hepatocytes, the method comprising the step of culturing one or more hepatocyte in contact with an extracellular matrix (ECM) in the presence of an expansion medium comprising a basal medium for human cells to which is added:
one or more Wnt signaling activator, one or more receptor tyrosine kinase ligand, and one or more epithelial phenotype stabilizing agent.

2. The method of claim 1, wherein the expansion medium further comprises one or more cell survival agent.

3. The method of claim 1 or 2, wherein the expansion medium further comprises one or more cell proliferation agent.

4. The method of any one of claims 1-3, wherein the one or more Wnt signaling activator comprises R-spondin 1, R-spondin 2, R-spondin 3, R-spondin 4, Wnt3a, or a combination of any of the foregoing.

5. The method of claim 4, wherein the one or more Wnt signaling activator comprises R-spondin 1 and Wnt3a.

6. The method of any one of claims 1-5, wherein the receptor tyrosine kinase is an epidermal growth factor (EGF) receptor tyrosine kinase or a fibroblast growth factor (FGF) receptor tyrosine kinase.

7. The method of claim 6, wherein the one or more receptor tyrosine kinase ligand comprises an EGF, an FGF, a hepatocyte growth factor (HGF), a transforming growth factor-alpha (TGF-alpha), or a combination of any of the foregoing.

8. The method of claim 7, wherein the one or more receptor tyrosine kinase ligand comprises human EGF, FGF-7, FGF-10, HGF, TGF-alpha, or a combination of any of the foregoing.

9. The method of claim 8, wherein the one or more receptor tyrosine kinase ligand comprises human EGF, FGF-7, FGF-10, HGF, and TGF-alpha.

10. The method of any one of claims 1-9, wherein the epithelial phenotype stabilizing agent is a TGF-beta inhibitor.

11. The method of claim 10, wherein the TGF-beta inhibitor is an activin receptor-like kinase (ALK) 5 inhibitor.

12. The method of claim 11, wherein the ALK5 inhibitor is A83-01.

13. The method of any one of claims 1-12, wherein the epithelial phenotype stabilizing agent further comprises a corticosteroid.

14. The method of claim 13, wherein the corticosteroid is hydrocortisone.

15. The method of any one of claims 1-14, wherein the expansion medium comprises N-acetylcysteine, nicotinamide, a Rho kinase inhibitor, or any combination of the foregoing.

16. The method of any one of claims 1-14, wherein the expansion medium does not comprise a Rho kinase inhibitor.

17. The method of claim 15 or 16, wherein the Rho kinase inhibitor is Y-27632.

18. The method of any one of claims 1-17, wherein the ECM comprises a collagen, a laminin, or both a collagen and a laminin.

19. The method of claim 18, wherein the collagen is collagen-I or collagen-IV.

20. The method of claim 18, wherein the laminin is laminin-111, laminin-211, laminin-221, laminin-332, laminin-411, laminin-421, laminin-511, or laminin-521.

21. The method of any one of claims 1-20, wherein the culturing step is performed on a surface.

22. The method of claim 21, wherein the surface is a two-dimensional surface.

23. The method of claim 22, wherein the two-dimensional surface comprises a surface area of between 9.5 cm² to 10,000 cm².

24. The method of claim 22, wherein the two-dimensional surface is coated with the ECM.

25. The method of any one of claims 21-24, wherein the hepatocytes are adherently attached to the surface during the culturing step.

26. The method of any one of claims 1-25, wherein the expansion medium further comprises a B27 supplement, an N2 supplement, or a combination thereof.

27. The method of claim 26, wherein the B27 supplement does not contain vitamin A.

28. The method of any one of claims 1-27, wherein the expansion medium does not contain a Notch inhibitor or a Notch agonist.

29. The method of any one of claims 1-28, wherein the expansion medium does not contain gastrin.

30. The method of any one of claims 1-29, wherein the expansion medium further comprises an amino acid supplement.

31. The method of claim 30, wherein the amino acid supplement comprises a non-essential amino acid (NEAA) supplement.

32. The method of any one of claims 1-31, wherein the expansion medium further comprises a serum replacement component.

33. The method of claim 32, wherein the serum replacement component comprises KNOCKOUT™ Serum Replacement (KOSR), human platelet lysate, human serum, or bovine serum.

34. The method of any one of claims 1-33, wherein the culturing comprises culturing the cells under hypoxic conditions or in the presence of a hypoxia mimetic.

35. The method of claim 34, wherein the culturing is performed under hypoxic conditions.

36. The method of claim 34 or 35, wherein the culturing under hypoxic conditions comprises culturing the cells at an oxygen level of between 1% to 19%.

37. The method of claim 36, wherein the oxygen level is between 1% to 10%.

38. The method of claim 37, wherein the oxygen level is 5%.

39. The method of any one of claims 1-38, wherein the culturing step comprises expanding plated cells (step P0) and a first passage of expanded cells (step P1).

40. The method of claim 39, wherein the P0 step has a duration of between 7 to 16 days.

41. The method of claim 40, wherein the P0 step has a duration of 11 days.

42. The method of claim 40, wherein the P0 step has a duration of 13 days.

43. The method of any one of claims 39-42, wherein the P1 step has a duration of between 7 to 20 days.

44. The method of claim 43, wherein the P1 step has a duration of 11 days.

45. The method of claim 43, wherein the P1 step has a duration of 13 days.

46. The method of any one of claims 39-45 wherein the P0 step comprises seeding the hepatocytes at a density of between 200 to 13,333 cells/cm².

47. The method of claim 46, wherein the P0 step comprises seeding the hepatocytes at a density of 667 cells/cm².

48. The method of any one of claims 39-47, wherein the P1 step comprises seeding the hepatocytes at a density of between 333 to 13,333 cells/cm².

49. The method of claim 48, wherein the P1 step comprises seeding the hepatocytes at a density of 1,333 cells/cm².

50. The method of any one of claims 39-49, wherein the expansion medium comprises a serum replacement component, and the concentration of the serum replacement component is varied over the duration of the culturing step.

51. The method of claim 50, wherein the concentration of the serum replacement component is 1% (v/v) on Day 0 of the P0 step and remains 1% (v/v) until the concentration of the serum replacement component is increased.

52. The method of claim 51, wherein the concentration of the serum replacement is raised to 5% (v/v) (i) when the cell density reaches between 15% to 30% confluency or (ii) between Day 3 to Day 7 of the P0 step.

53. The method of claim 52, wherein the concentration of the serum replacement is raised to 5% (v/v) on Day 5 of the P0 step.

54. The method of claim 50, wherein the concentration of the serum replacement component is 5% (v/v) on Day 0 of the P0 step.

55. The method of any one of claims 51-54, wherein the concentration of the serum replacement is raised to 10% (v/v) (i) when the cell density reaches between 40% to 60% confluency or (ii) between Day 7 to Day 13 of the P0 step.

56. The method of claim 55, wherein the concentration of the serum replacement is raised to 10% (v/v) on Day 9 of the P0 step.

57. The method of any one of claims 51-56, wherein the concentration of the serum replacement component is 1% (v/v) on Day 0 of the P1 step.

58. The method of claim 57, wherein the concentration of the serum replacement is raised to 5% (v/v) (i) when the cell density reaches between 15% to 30% confluency or (ii) between Day 3 to Day 7 of the P1 step.

59. The method of claim 58, wherein the concentration of the serum replacement is raised to 5% (v/v) on Day 5 of the P1 step.

60. The method of any one of claims 51-56, wherein the concentration of the serum replacement component is 5% (v/v) on Day 0 of the P1 step and remains 5% (v/v) until the concentration of the serum replacement component is increased.

61. The method of any one of claims 51-60, wherein the concentration of the serum replacement is raised to 10% (v/v) (i) when the cell density reaches between 40% to 60% confluency or (ii) between Day 5 to Day 13 of the P1 step.

62. The method of claim 61, wherein the concentration of the serum replacement is raised to 10% (v/v) on Day 7 of the P1 step.

63. The method of claim 61, wherein the concentration of the serum replacement is raised to 10% (v/v) on Day 9 of the P1 step.

64. The method of any one of claims 1-63, wherein the method further comprises determining, following the culturing step, the expression profile of the hepatocytes.

65. The method of claim 64, wherein the duration of the culturing step is for at least 3 days.

66. The method of claim 65, wherein, following the culturing step, the expression profile of the hepatocytes includes expression of one or more protein selected from hepatocyte nuclear factor 4 alpha (HNF4α), leucine rich repeat containing G protein-coupled receptor 5 (LGR5), keratin 18 (CK18), and albumin by at least 80% of the hepatocytes.

67. The method of claim 65, wherein, following the culturing step, the expression profile of the hepatocytes includes expression of Ki67 by at most 15% of the hepatocytes.

68. The method of claim 65, wherein, following the culturing step, the expression profile of the hepatocytes includes expression of Ki67 by at least 15% of the hepatocytes.

69. The method of claim 65, wherein, following the culturing step, the expression profile of the hepatocytes includes downregulation of one or more proteins characteristic of mature cell state and upregulation of one or more fetal/hepatic progenitor/cholangiocyte markers by at least 10% compared to overnight plated control hepatocytes.

70. The method of claim 69, wherein the mature hepatocyte cell state proteins are NR112, Urea cycle enzymes, Cyp3a4, Cyp1a2, ABCG2, ABCC2, ABCB11, SR-B1, or SLC10A1.

71. The method of claim 69, wherein the fetal/hepatic progenitor/cholangiocyte markers are AFP, Cyp3a7, EPCAM, LGR5, KRT7, KRT19, or AQP1.

72. The method of claim 65, wherein, following the culturing step, the expression profile of the hepatocytes includes expression of one or more genes selected from HNF4α, LGR5, CK18, and ALB by at least 80% of the hepatocytes.

73. The method of claim 65, wherein, following the culturing step, the expression profile of the hepatocytes includes expression of Ki67 by at most 15% of the hepatocytes.

74. The method of claim 65, wherein, following the culturing step, the expression profile of the hepatocytes includes expression of Ki67 by at least 15% of the hepatocytes.

75. The method of any one of claims 1-74, wherein, following the culturing step, the expression profile of the hepatocytes includes decreased expression of urea cycle enzymes.

76. The method of any one of claims 1-75, wherein, following the culturing step, the hepatocyte yield is at least 5×10³ hepatocytes per 1 cm².

77. The method of any one of claims 1-76, wherein, following the culturing step, the hepatocyte yield expands by at least 2-fold within 14 days of culturing.

78. The method of any one of claims 1-77, wherein, following the culturing step, the hepatocyte yield expands by at least 500-fold within 30 days of culturing.

79. The method of claim 78, wherein, following the culturing step, the hepatocyte yield expands by at least 500-fold within 24 days of culturing.

80. The method of claim 78, wherein the hepatocyte yield expands by between 500-fold to 2000-fold within 30 days of culturing.

81. The method of claim 78, wherein the hepatocyte yield expands by between 500-fold to 2000-fold within 24 days of culturing.

82. The method of any one of claims 1-81, wherein the method further comprises maturing the hepatocytes in a maturation medium comprising a basal medium for human cells to which is added one or more hepatocyte maturation supplements.

83. A method for maturing a population of hepatocytes, the method comprising the step of maturing an expanded hepatocyte population in the presence of a maturation medium comprising a basal medium for human cells to which is added one or more hepatocyte maturation supplements.

84. The method of claim 82 or 83, wherein the maturation step begins immediately following hepatocyte expansion.

85. The method of claim 82 or 83, wherein the maturation step does not begin immediately following hepatocyte expansion.

86. The method of any one of claims 82-85, wherein the maturation step has a duration of between 3 and 12 days.

87. The method of any one of claims 82-86, wherein the maturation step has a duration of 7 days.

88. The method of any one of claims 82-87, wherein the maturation basal medium is LONZA™ HCM™, William's E, or HepatoZYME-SFM.

89. The method of any one of claims 82-99, wherein the one or more maturation supplements comprises an antibiotic, 4-(2-hydroxyethyl)-1-piperazineethanesulfonic acid (HEPES), GLUTAMAX™, ITS, a Notch inhibitor, an EGFR inhibitor, Oncostatin M, an anti-oxidant, a glucocorticoid, a pregnane X receptor (PXR) activator, a bile acid, a cAMP or cAMP analog, cholesterol, a thyroid hormone, a serum replacement component, or any combination of the foregoing.

90. The method of claim 89, wherein the Notch inhibitor is Compound E, Gamma Secretase Inhibitor XX, or a combination thereof.

91. The method of claim 89, wherein the antibiotic is penicillin, streptomycin, or a combination thereof.

92. The method of claim 89, wherein the EGFR inhibitor is erlotinib HCl.

93. The method of claim 89, wherein the anti-oxidant is vitamin C.

94. The method of claim 89, wherein the glucocorticoid is dexamethasone, hydrocortisone, or a combination thereof.

95. The method of claim 89, wherein the PXR activator is vitamin K2.

96. The method of claim 89, wherein the bile acid is lithocholic acid, urso deoxycholic acid, or a combination thereof.

97. The method of claim 89, wherein the CAMP analog is 8 bromo CAMP, forskolin, or a combination thereof.

98. The method of claim 89, wherein the thyroid hormone is T3.

99. The method of claim 89, wherein the serum replacement component is ITS, KOSR, Trace Elements A, Trace Elements B, or a combination thereof.

100. The method of any one of claims 82-99, wherein the maturation medium lacks one or more of R-spondin 1/Wnt3a, Epidermal Growth Factor (EGF), Transforming Growth Factor α (TGFα), N-acetyl cysteine, Nicotinamide, B27 supplement, N2 supplement, Fibroblast Growth Factor 7 (FGF7), and Fibroblast Growth Factor 10 (FGF10).

101. A kit comprising an expansion medium comprising a basal medium for human cells to which is added one or more Wnt signaling activator, one or more receptor tyrosine kinase ligand, and one or more epithelial phenotype stabilizing agent, wherein the kit further comprises a package insert instructing a user of the kit to culture one or more hepatocyte in accordance with the method of any one of claims 1-81.

102. An expansion medium comprising a basal medium for human cells to which is added one or more Wnt signaling activator, one or more receptor tyrosine kinase ligand, and one or more epithelial phenotype stabilizing agent.

103. The expansion medium of claim 102, wherein the expansion medium further comprises one or more cell survival agent.

104. The expansion medium of claim 102 or 103, wherein the expansion medium further comprises one or more cell proliferation agent.

105. The expansion medium of any one of claims 102-104, wherein the one or more Wnt signaling activator comprises R-spondin 1, R-spondin 2, R-spondin 3, R-spondin 4, Wnt3a, or a combination of any of the foregoing.

106. The expansion medium of claim 105, wherein the one or more Wnt signaling activator comprises R-spondin 1 and Wnt3a.

107. The expansion medium of any one of claims 102-106, wherein the receptor tyrosine kinase ligand is an EGF or an FGF.

108. The expansion medium of claim 107, wherein the receptor tyrosine kinase ligand comprises an EGF, an FGF, an HGF, a TGF-alpha, or a combination of any of the foregoing.

109. The expansion medium of claim 107, wherein the receptor tyrosine kinase ligand comprises human EGF, FGF-7, FGF-10, HGF, or TGF-alpha.

110. The expansion medium of anyone of claims 102-109, wherein the epithelial phenotype stabilizing agent is a TGF-beta inhibitor.

111. The expansion medium of claim 110, wherein the TGF-beta inhibitor is an ALK5 inhibitor.

112. The expansion medium of claim 111, wherein the ALK5 inhibitor is A83-01.

113. The expansion medium of any one of claims 102-112, wherein the epithelial phenotype stabilizing agent further comprises a corticosteroid.

114. The expansion medium of claim 113, wherein the corticosteroid is hydrocortisone.

115. The expansion medium of any one of claims 102-114, wherein the expansion medium comprises N-acetylcysteine, nicotinamide, or a Rho kinase inhibitor.

116. The expansion medium of any one of claims 102-115, wherein the expansion medium does not comprise a Rho kinase inhibitor.

117. The expansion medium of claim 115 or 116, wherein the Rho kinase inhibitor is Y-27632.

118. The expansion medium of any one of claims 102-117, wherein the expansion medium further comprises a B27 supplement, an N2 supplement, or a combination thereof.

119. The expansion medium of claim 118, wherein the B27 supplement does not contain vitamin A.

120. The expansion medium of any one of claims 102-119, wherein the expansion medium does not contain a Notch inhibitor or a Notch agonist.

121. The expansion medium of any one of claims 102-120, wherein the expansion medium does not contain gastrin.

122. The expansion medium of any one of claims 102-121, wherein the expansion medium further comprises an amino acid supplement.

123. The expansion medium of claim 122, wherein the amino acid supplement comprises an NEAA supplement.

124. The expansion medium of any one of claims 102-123, wherein the expansion medium further comprises a serum replacement component.

125. The expansion medium of claim 124, wherein the serum replacement component comprises KOSR, human platelet lysate, human serum, or bovine serum.

126. A tissue culture vessel comprising the expansion medium of any one of claims 102-125.

127. An incubator comprising the tissue culture vessel of claim 126, wherein the incubator maintains the tissue culture vessel under hypoxic conditions.

128. A kit comprising a maturation medium comprising a basal medium for human cells to which is added one or more maturation supplements, wherein the kit further comprises a package insert instructing a user of the kit to mature one or more hepatocyte in accordance with the method of any one of claims 82-100.

129. A maturation medium comprising a basal medium for human cells to which is added one or more hepatocyte maturation supplements.

130. The maturation medium of claim 129, wherein the maturation basal medium is LONZA™ HCM™, William's E, or HepatoZYME-SFM.

131. The maturation medium of claim 129, wherein the one or more maturation supplements comprises an antibiotic, 4-(2-hydroxyethyl)-1-piperazineethanesulfonic acid (HEPES), GLUTAMAX™, ITS, a Notch inhibitor, an EGFR inhibitor, Oncostatin M, an anti-oxidant, a glucocorticoid, a pregnane X receptor (PXR) activator, a bile acid, a cAMP or cAMP analog, cholesterol, a thyroid hormone, a serum replacement component, or any combination of the foregoing.

132. The maturation medium of claim 131, wherein the Notch inhibitor is Compound E, Gamma Secretase Inhibitor XX, or a combination thereof.

133. The maturation medium of claim 131, wherein the antibiotic is penicillin, streptomycin, or a combination thereof.

134. The maturation medium of claim 131, wherein the EGFR inhibitor is erlotinib HCl.

135. The maturation medium of claim 131, wherein the anti-oxidant is vitamin C.

136. The maturation medium of claim 131, wherein the glucocorticoid is dexamethasone, hydrocortisone, or a combination thereof.

137. The maturation medium of claim 131, wherein the PXR activator is vitamin K2.

138. The maturation medium of claim 131, wherein the bile acid is lithocholic acid, urso deoxycholic acid, or a combination thereof.

139. The maturation medium of claim 131, wherein the CAMP analog is 8 bromo CAMP, forskolin, or a combination thereof.

140. The maturation medium of claim 131, wherein the thyroid hormone is T3.

141. The maturation medium of claim 131, wherein the serum replacement component is ITS, KOSR, Trace Elements A, Trace Elements B, or a combination thereof.

142. The maturation medium of any one of claims 129-141, wherein the maturation medium lacks one or more of R-spondin 1/Wnt3a, Epidermal Growth Factor (EGF), Transforming Growth Factor α (TGFα), N-acetyl cysteine, Nicotinamide, B27 supplement, N2 supplement, Fibroblast Growth Factor 7 (FGF7), and Fibroblast Growth Factor 10 (FGF10).
